SEQUENCE LISTING (14 sequences)

SEQ ID NO: 1 - protein, 1261 aa, P. aeruginosa
  MISC_FEATURE (1)..(1261): Sequence of NarG gene
MSHLLDRLQFFKKKQGEFADGHGETSNESRAWEGAYRQRWQHDKIVRSTHGVNCTGSCSWKIYVKNGLITWETQQTDYPRTRPDLPNHEPRGCPRGASYSWYIYSANRLKYPKVRKPLLKLWREARAQHGDPVNAWASIVEDAAKAKSYKSQRGLGGFVRSSWDEVTEIIAAANVYTAKTYGPDRVIGFSPIPAMSMVSYAAGARYLSLIGGVCLSFYDWYCDLPPASPQIWGEQTDVPESADWYNSSYIIAWGSNVPQTRTPDAHFFTEVRYKGTKTVSITPDYSEVAKLTDLWLNPKQGTDAALGMAFGHVILKEFHLDRPSAYFVDYCRQYTDMPMLVLLEEHAGGAFKPTRYLRAADLADNLGQDNNPEWKTIAYDERSGGLVSPTGAIGYRWGESGKWNIAELDGRSGDQTRLQLSLLDGPEHACEVAFPYFAGQEHPHFKGVANDEVLLRRVPFREIVAADGKRLRVATVYDLQMANYSIDRGLGGDNVATSYEDADTPYTPAWQERITGVPAARATQVAREFADSADKTRGKAMVIIGAAMNHWYHMDMNYRAVINMLMMCGCIGQSGGGWAHYVGQEKLRPQTGWAPLAFGLDWSRPPRQMNGTSFFYLHSSQWRHEKLSMHEVLSPLADASRFAEHALDYNIQAERLGWLPSAPQLNRNPLRIAAEAEAAGLPVADYVVRELKSGGLRFASESPDDPQNFPRNMFIWRSNLLGSSGKGHEYMLKYLLGAKNGVMNDDLGKAGGPRPTEVDWVDDGAEGKLDLVTTLDFRMSSTCMYSDIVLPTATWYEKDDLNTSDMHPFIHPLSAATDPAWEAKSDWEIYKAIAKKFSAVAEGHLGVEQDLVTVPLLHDTPTELAQPFGGDGHDWKKGECEPMPGRNLPTLHLVERDYPNVYRKFTSLGPLLDKLGNGGKGIGWNTEKEVKLVGDLNHRVVESGVSQGRPRIDSAIDAAEVVLALAPETNGQVAVKAWEALSKITGREHAHLALPKEDEKIRFRDIQVQPRKIISSPTWSGLEDEHVSYNAGYTNVHELIPWRTITGRQQFYQDHPWMQAFGEGFVSYRPPVNTRTTEKLLNRKPNGNPEITLNWITPHQKWGIHSTYSDNLLMLTLSRGGPIIWLSEHDAAKAGIVDNDWVEVFNANGAATCRAVVSQRVKDGMVMMYHAQERIVNVPGSETTGTRGGHHNSVTRVVLKPTHMIGGYAQQAWGFNYYGTVGCNRDEFVVVRKMSKVDWLDEPRHGGLGGDALPQPLPQDI

SEQ ID NO: 2 - DNA, 3786 bp, P. aeruginosa
  misc_feature (1)..(3786): sequence of narG gene
atgagtcacctgctcgaccgcctgcagttcttcaagaagaagcagggcgaattcgccgatggccacggcgagaccagcaacgagagccgcgcctgggaaggtgcctaccggcagcgctggcagcacgacaagatcgtgcgctccacccacggggtgaactgcaccggctcctgctcctggaagatctacgtgaagaacggcctgatcacctgggaaacccagcagaccgactacccgcgcacccgtccggacctgcccaaccacgagccgcgcggctgcccgcgcggggccagctattcctggtacatctacagcgccaaccgcctgaagtacccgaaggtgcgcaagccgttgctcaagctctggcgcgaggcgcgggcgcagcacggcgacccggtgaacgcctgggccagcatcgtcgaggacgccgccaaggcgaagagctacaagagccagcgcggcctgggcggcttcgtccgttccagctgggacgaggtcaccgagatcatcgccgcggccaacgtctacaccgccaagacctacggtccggaccgggtgatcggcttctcgccgatcccggccatgtcgatggtcagctacgccgccggcgcccgctacctgtcgctgatcggcggggtctgcctgagcttctacgactggtactgcgacctgccgccggccagcccgcagatctggggcgagcagaccgacgtgccggagtcggccgactggtacaactccagctacatcatcgcctggggctccaacgtgccgcagacgcggaccccggacgcgcacttcttcaccgaggtgcgctacaagggcaccaagaccgtctccatcaccccggactattccgaggtggccaagctcaccgacctctggctcaaccccaagcagggcaccgacgccgcgctgggcatggccttcggtcacgtgatcctgaaggaattccacctcgaccggccgagcgcctacttcgtcgactactgccgccagtacaccgacatgccgatgctggtgttgctggaagaacacgccggcggcgcgttcaagccgacccgctacctgcgcgccgccgacctggcggacaacctcggccaggacaacaaccccgagtggaagaccatcgcctacgacgagcgcagcggcgggctggtctcgcccaccggcgccatcggctatcgctggggcgagtcaggcaagtggaacatcgccgagctggacggcaggagcggtgaccagacgcgcctgcaactgtcgctgctcgatggcccggaacatgcctgcgaggtggccttcccgtatttcgccgggcaggagcacccgcacttcaagggcgtcgccaacgacgaggtactgctgcgccgggtgccgttccgcgagatcgtcgcggcggacggcaagcgcctgcgggtggccaccgtctacgacctgcagatggccaactacagcatcgaccgcggcctgggcggcgacaacgtggcgacctcctacgaggacgccgacacgccctataccccggcctggcaggagcgcatcaccggcgttccggcggcgcgcgcgacgcaggtcgcccgcgagttcgccgacagcgccgacaagacccgcggcaaggcgatggtgatcatcggcgcggcgatgaaccactggtaccacatggacatgaactaccgcgcggtcatcaacatgctgatgatgtgcggctgcatcggccagagcggcggcggctgggcgcactatgtcggccaggagaagctgcgcccgcagaccggctgggcgccgctggccttcggcctggactggagccggccgccgcggcagatgaacggcaccagcttcttctacctgcacagctcgcaatggcgccacgagaagctgtcgatgcacgaggtgctgtcgccgctggccgacgccagccgcttcgccgaacacgccctggactacaacatccaggccgaacgcctcggctggctgccgtcggcgccgcaactgaaccgcaacccgctgcgcatcgccgccgaggccgaggccgccggcctgccggtcgccgactacgtggtgcgcgaactgaagagcggcggcctgcgcttcgccagcgaatcgccggacgatccgcagaacttcccgcgcaacatgttcatctggcgctccaacctgctgggctcctccggcaagggccacgagtacatgctcaagtacctgctcggggcgaagaacggggtgatgaacgatgacctcggcaaggccggcggtccgcgtcccaccgaggtcgactgggttgacgacggtgccgagggcaagctcgacctggtcaccaccctggacttccgcatgtcctccacctgcatgtactcggacatcgtcctgccgaccgctacctggtacgagaaggacgacctcaacacctccgacatgcaccccttcatccatccgctgtcggcggccaccgatccggcctgggaagccaagagcgactgggagatctacaaggccatcgccaagaagttctccgccgtcgccgaaggccacctcggcgtggagcaggacctggtcacggtgccgctgctgcacgacacccccaccgagctggcgcagccgttcggcggcgacggccatgactggaagaagggcgagtgcgagccgatgccgggacgcaacctgccgacgctgcacctggtcgagcgcgactacccgaacgtctaccgcaagttcacctcgctcggtccgctgctggacaagctgggcaacggcggcaagggcatcggctggaacaccgagaaggaagtgaagctggtcggcgacctcaaccatcgcgtcgtcgagagcggcgtcagccagggccgcccgcgcatcgacagcgccatcgacgccgctgaggtggtcctcgccctggctccggaaaccaacggccaggtcgcggtcaaggcctgggaagcgctgtcgaagatcaccggccgcgagcatgcccacctggcgctgcccaaggaagacgagaagatccgcttccgcgacatccaggtgcagccgcgcaagatcatctccagcccgacctggtccggcctcgaggacgagcacgtcagctacaacgccggctacaccaacgtccacgagctgatcccgtggcgcaccatcaccggtcgccagcagttctaccaggaccacccgtggatgcaggcgttcggcgaaggcttcgtcagctaccggccgccggtcaacacccggaccaccgagaaactgttgaacaggaagcccaacggcaacccggagatcaccctgaactggatcaccccgcaccagaaatggggcatccactccacctacagcgacaacctgctgatgctcaccctgtcgcgcggcggtccgatcatctggctcagcgagcacgacgcggccaaggccgggatcgtcgataacgactgggtcgaggtgttcaacgccaacggcgcggcgacctgccgcgcggtggtcagccagcgggtcaaggacggcatggtgatgatgtaccacgcccaggaacgcatcgtgaacgtacccggcagcgagaccaccggcacccgtggcggccaccacaactcggtgacccgcgtggtgctcaagccgacccacatgatcggcggctacgcccagcaggcctggggcttcaactactacggcacggtcggctgcaaccgcgacgagttcgtcgtggtgcgcaagatgagcaaggtcgactggctggacgaaccccgccacggcggactcggcggcgacgccctgccgcaaccgctgccccaggacatttga

SEQ ID NO: 3 - DNA, 1542 bp, P. aeruginosa
  misc_feature (1)..(1542): sequence of narH gene
atgaaaattcgttcgcaagtcggcatggtgctgaacctcgacaagtgcattggttgccacacctgctcgatcacctgcaagaacgtctggaccagccgcgaaggcatggagtacgcctggttcaacaacgtcgagaccaagcccgggatcggctacccgaaggaatgggaaaaccaggagaagtggaagggcggctgggtgcgcgcggcggacggttcgatccgcccgcgcatcggcggcaagttccgcgtgctggcgaacatcttcgccaacccggacctgcccgagatcgacgactactacgaaccgttcgacttcgattaccagcacctgcataccgcgcccaaggccgagcaccagccggtggcgcgcccgcgctcgctggtctccgggcagcgcatggagaagatcgagtggggcccgaactgggaggagatcctcggcaccgagttcgccaagcggcgcaaggacaagaacttcgaccaggtccaggcggacatctacggtgagtacgagaacaccttcatgatgtacctgccgcgcctctgcgagcactgcctgaacccggcgtgcgtggcgtcctgcccgagcggggcgatctacaagcgcgaggaggacggcatcgtcctgatcgaccaggacaagtgccgcggctggcggatgtgcatctccggctgcccgtacaagaagatctacttcaactggaagagcggcaagtccgagaagtgcatcttctgctacccgcgcatcgaggccggccagcccactgtctgctcggagacctgcgtcgggcgcatccgctacctcggcgtgctgctctacgacgccgaccgcatccacgaagtggccagttgcgagaacgagcgcgagctgtacgagaagcaactggagatcttcctcgatccgttcgacccggcggtgatcgcccaggcgcgcaaggacggggtggccgacagcgtcatcgaggcggcgcagaagtcgccggtgtacaagctggcgatggactggaagctggccctgccgctgcacccggaataccgcacgctgccgatggtctggtacgtgccaccgctgtcgccgatccagaacgccgccgccgaggggcacatcggcagcgacggggtgatcccggacgtggagtcgctgcgcatccccgtgcagtacctggccaacctgctcaccgccggcgacaccgcgccggtgctgctggcgctcaagcgcctgctggcgatgcgcgcctacaagcgcgccgagcacgtcgaaggccgccaggacctggaggtgctggccaaggtcgggttgagcgtggagcaggtggaggagatgtaccgctacctggccatcgccaactacgaggatcgcttcgtgatccccagcgcgcaccgcgaggaagcgctttccgatgccttcgccgagcgttccggctgcggcttcagcttcggcaacggctgttccggcggcagcaactccgccgtcaacctgttcggcggcaagccgaccaaccgccgcgacgtgatccaggtcgtgcagatccaggagtga

SEQ ID NO: 4 - DNA, 741 bp, P. aeruginosa
  misc_feature (1)..(741): sequence of narJ gene
atgaacgatcacagccaactgttccgcctgctcgccctgctgctcgactatccacgcgccgagctgcgcgaggagagcctcggcctgcatgcgctgatccgcacctgcgaactgccggaagcgctgcgcgacggcctcgcggcgctgctcaacgagctctgccagggcgacctgctggacgtccaggcgcgctacgacggtctcttcgagcgcggccgctcggtctcgctgctgctcttcgagcacgtccacggcgagagccgcgaccgtggccaggcgatggtcgacctgctcgaccgctataccggggccggcctgcagatcgacgtaccggagctgccggactacctgccgctgtacctcgaatacctgtcgctgctgccgttcgcggcggccagcgaagggctcgccgaagtggcgcacatcctcggcctgctggcgctgcgcctggaggaacgcggcagcgcctacgcggcgattttcgaggcgttgctggaactcggcggcgagcgcccggacctcggcgcgttgcgtcgcgaccaggcccaggaacagcgcgacgacagcctggaggccatcgaccgggcctgggaggaaaccccggtgagcttcaccgaccctgccggcggttgcccgtcgagcagcggccgccgtccgacggcgtccaccgaacaaccattgcaatgggtcgcccagccggtaccgcagatgcagtaccgcgcggcccgcgaaggagtctga

SEQ ID NO: 5 - DNA, 684 bp, P. aeruginosa
  misc_feature (1)..(684): sequence of narI gene
atgtcgaccaatcttctgttcttcgggatctatccctatgtcgcgctgctgatctgcctggtcggcagctgggcgcgcttcgacctctcgcagtacacctggaaggccggttccagccagatgctcagcaagaagggcatgcgggtatacagcaacctgttccacgtcggcgtgctgttcatcctcgccggccacttcgtcggcctgctgaccccggcctcggtctaccaccacctgatcagcaccgagaacaagcaactgctggcgatggtctccggcggcttcttcggcgtgctctgcttcatcggcctgagcggactgatcctgcgccgcctgaccgacgcgcgggtgcgcgccaccggcaacgcctctgacctgatgatcctgctggtgctctacgcccagctgatcctcggcctctccaccatcgtcgcctcgacccatcacatggatggctcggtgatggtgatgctcgccgactgggcccaggccatcgtcaccctgcgtccgctggcggcggccgaagccatcgcgccggtgggcctggtctacaagctgcacgtcggcctgggcctgaccctgttcgtgctgttccccttcacccgcctggtgcacatcgtcagcgcgccggtgtggtacctgggccggcgctaccagatcgtgcgtcagaaacgtcctgcctga

SEQ ID NO: 6 - protein, 834 aa, P. aeruginosa
  MISC_FEATURE (1)..(834): sequence of NapA gene
MNLTRREFAKANAAAIAAAAAGLPILVRASNLVTEADVTSLVWNKAPCRFCGTGCSVMVATRDGQVVATHGDIKAEVNRGINCVKGYFLSKIMYGSDRLTRPLLRMKDGKFDKQGEFQPISWEQAFDIMAEKFKAALKAKGPESVGMFGSGQWTVWEGYAANKLFKAGLRSNNIDPNARHCMASAVMGFMRSFGMDEPMGCYDDIEATDSFVLWGSNMAEMHPVLWSRVTDRRLSAPQVKVAVLSTFEHRSFELADLPMVFKPQTDLIILNYIANHIIESGAVNRDFVERHVRFAHGAEDIGYGLRPDDPLEKKAKNADKANTWSDIDFKAFAEFVKPYTLERTARESGVPAERLKALAELYADPKRKVVSFWTMGFNQHTRGVWANNLIYNIHLLTGKISEPGNSPFSLTGQPSACGTAREVGTFSHRLPADLVVTNPKHRETAEKIWKVPAGTIQEKVGFHAVQQSRMLNDGVLNVYWTQVSNNMQAGPNVMQEVLPGWRNPDNFVIVSDVYPTVSAQAADLILPSAMWVEKEGAFGNAERRTQFWHQLVKAPGEAKSDLWQLVEFSKRFTTDEVWPAELLAKAPELKGKTLYDVLFRNGQVDRFPASDLAKGYANDEVDAFGFYIQKGLFEEYAAFGRGHGHDLAPFDAYHEARGLRWPVVDGKETRWRYREGYDPYVSKGSGVQFYGYPDKKAIVFALPYEPPAEAPDQDYPFWLATGRVLEHWHTGSMTARVPELYKAVPDALVYMHPEDARQLKLRRGSEVKVVSRRGEIRARVETRGRNKPPQGLVFVPFFDANKLINKVTLDATDPISKQTDYKKCAVRIELLNLA

SEQ ID NO: 7 - DNA, 60 bp, Artificial Sequence;
  Synthetic Polynucleotide
ccatacccgtttttttgggctagcgaattcgagctcaggaggagatcaagatgagtcacc

SEQ ID NO: 8 - DNA, 60 bp, Artificial Sequence;
  Synthetic Polynucleotide
gcaaattctgttttatcagaccgcttctgcgttctgatttaaggttcaggcaggacgttt

SEQ ID NO: 9 - DNA, 60 bp, Artificial Sequence;
  Synthetic Polynucleotide
ccatacccgtttttttgggctagcgaattcgagctcaggaggctgggccaatgaacgaac

SEQ ID NO: 10 - DNA, 60 bp, Artificial Sequence;
  Synthetic Polynucleotide
gcaaattctgttttatcagaccgcttctgcgttctgatttaagcgctaccagcccttcac

SEQ ID NO: 11 - DNA, 40 bp, Artificial Sequence;
  Synthetic Polynucleotide
taaaacgacggccagtgccacgtactgggtgttcgccctg

SEQ ID NO: 12 - DNA, 30 bp, Artificial Sequence;
  Synthetic Polynucleotide
cgcgcagggtcttgatctcctcacccggtc

SEQ ID NO: 13 - DNA, 30 bp, Artificial Sequence;
  Synthetic Polynucleotide
ggagatcaagaccctgcgcggccggcgcat

SEQ ID NO: 14 - DNA, 40 bp, Artificial Sequence;
  Synthetic Polynucleotide
catgattacgaattcgagctgctggcgcggcaggaagcgc

(19) United States
(12) Patent Application Publication
(10) Pub. No.: US 2019/0142864 A1
(43) Pub. Date: May 16, 2019

(54) METHODS AND SYSTEMS, FOR INTERFERING WITH VIABILITY OF BACTERIA AND RELATED ANTIMICROBIALS AND COMPOSITIONS

(71) Applicants: CALIFORNIA INSTITUTE OF TECHNOLOGY, Pasadena, CA (US); THE REGENTS OF THE UNIVERSITY OF CALIFORNIA, Oakland, CA (US)

(72) Inventors: Dianne K. NEWMAN, Pasadena, CA (US); John COATES, Walnut Creek, CA (US); Melanie A. SPERO, Pasadena, CA (US)

(21) Appl. No.: 16/157,885

(22) Filed: Oct. 11, 2018

Related U.S. Application Data
(60) Provisional application No. 62/571,009, filed on Oct. 11, 2017.

(51) Int. Cl. A61K 33/20; A61P 31/04; A61K 31/7036

(52) U.S. Cl. CPC A61K 33/20; A61K 31/7036; A61P 31/04

(57) ABSTRACT

Provided herein are methods, systems, and related compounds and compositions suitable for reducing antibiotic resistance and/or the survivability of Nar (cytoplasmic nitrate reductase)-and/or Nap (periplasmic nitrate reductase)-containing bacteria.

### CROSS-REFERENCE TO RELATED APPLICATION

The present application claims priority to U.S. Provisional Application No. 62/571,009, entitled “A New Therapeutic Strategy to Combat Diverse Chronic Infections” filed on Oct. 11, 2017, with docket number CIT 7310-P3, the contents of which are incorporated by reference in its entirety.

### STATEMENT OF GOVERNMENT GRANT

This invention was made with government support under Grant No. NIH 5R01HL117328-03 and Grant No. NIH 1R01AI127850-01A1 awarded by the National Institutes of Health. The government has certain rights in the invention.

### FIELD

The present disclosure relates to methods and systems for interfering with the viability of bacteria and related antimicrobials and compositions.

### BACKGROUND

Bacterial viability has been the focus of research in the field of biological analysis, in particular, when aimed at medical applications such as therapeutic or diagnostic applications.

Whether for pathological examination or for fundamental biology studies, several methods are commonly used for the detection of and interference with the viability of bacteria.

Although various methods, systems and compositions have been developed to interfere with, and in particular, to reduce bacterial viability to the extent of killing the bacteria. However, antibiotic resistance and additional defense mechanisms of the microorganism have made the development of methods, systems and compositions that are able to interfere with and in particular to inhibit bacterial viability particularly challenging.

### SUMMARY

Provided herein are methods, systems, and related compounds and compositions suitable for reducing antibiotic resistance and/or the survivability of bacteria. In several embodiments, the methods, systems and related compositions herein described are expected to be suitable to treat and/or prevent bacterial infection in vitro or in vivo caused by Nar (cytoplasmic nitrate reductase)-and/or Nap (periplasmic nitrate reductase)—containing bacteria.

According to a first aspect, a method and a system to interfere with viability of Nar-containing bacteria in a medium are described. The method comprises contacting the Nar-containing bacteria with an effective amount of chlorate alone or in combination with antibiotics and/or other antimicrobials for a time and under conditions to reduce survivability and/or antibiotic resistance of the bacteria. The system comprises a chlorate, antibiotics and/or antimicrobials for simultaneous combined or sequential use in the method to interfere with viability of Nar-containing bacteria herein described. In preferred embodiments, the administering of the chlorate is performed in absence of another chlorine oxyanion.

According to a second aspect, a method and a system are described for treating and/or preventing a bacterial infection by a Nar-containing bacterium in an individual. The method comprises administering to the individual an effective amount of chlorate alone or in combination with antibiotics and/or other antimicrobials. The administering performed in absence of another chlorine oxyanion. In particular, in some embodiments, administering of chlorate can be performed in combination with one or more antibiotics and/or other antimicrobials. In some embodiments, the bacterial infection is chronic infection. The system comprises chlorate, one or more antibiotics and/or antimicrobials for simultaneous combined or sequential use in the method for treating and/or preventing a bacterial infection by a Nar-containing bacterium in an individual herein described.

According to a third aspect, a method and a system to interfere with viability of Nap-containing bacteria in a medium are described. The method comprises contacting the Nap-containing bacteria with an effective amount of chlorate alone or in combination with antibiotics and/or other antimicrobials for a time and under conditions to reduce survivability and/or antibiotic resistance of the bacteria. The system comprises a chlorate, antibiotics and/or antimicrobials for simultaneous combined or sequential use in the method to interfere with viability of Nap-containing bacteria herein described. In preferred embodiments, the administering the chlorate is performed in absence of another chlorine oxyanion.

According to a fourth aspect, a method and a system are described for treating and/or preventing a bacterial infection by a Nap-containing bacterium in an individual. The method comprises administering to the individual an effective amount of chlorate alone or in combination with an antibiotic and/or other antimicrobial the administering performed in absence of another chlorine oxyanion. In particular, in some embodiments, administering of chlorate can be performed in combination with one or more antibiotics and/or other antimicrobials. In some embodiments, the bacterial infection is chronic infection.

The system comprises chlorate, one or more antibiotics and/or antimicrobials for simultaneous combined or sequential use in the method to for treating and/or preventing a bacterial infection by a Nap-containing bacterium in an individual herein described.

According to a fifth aspect, an antimicrobial is described, the antimicrobial comprising chlorate in an amount effective to reduce antibiotic resistance and/or survivability of Nar-containing bacteria in absence of another chlorine oxyanion. In some embodiments, the antimicrobial comprises a compatible vehicle, which can be a vehicle for effective administration and/or delivery of chlorate to an individual.

According to a sixth aspect, a composition is described. The composition comprises chlorate together with a compatible vehicle. In some embodiments, the composition can comprise one or more medium components as will be understood by a skilled person. In some embodiments the composition is a pharmaceutical composition and the compatible vehicle is a pharmaceutically compatible vehicle.

The antimicrobial compositions, methods and systems herein described, in several embodiments allow reducing antibiotic resistance and/or bacterial survivability of Nar-containing bacteria via Nar-mediated chlorate reduction that degrades chlorate into chlorite, surprisingly killing the Nar-containing bacteria at chlorate concentration non toxic for cells lacking Nar and/or Nap also present in the medium.

Accordingly, in some embodiments, antimicrobial compositions, methods and systems herein described, surprisingly allow to selectively target Nar-and/or Nap-containing bacteria in a medium while minimizing the interference with the viability of cells lacking Nar and/or Nap also possibly or known to be present in the medium.

In some embodiments, antimicrobial compositions, methods and systems herein described, surprisingly also allow to target the Nar- and/or Nap-containing bacteria in a medium in absence of any other chlorine oxyanions such as perchlorate, chlorite or hypochlorite or other chemical compounds which are known or expected to have toxicity effects on non-Nar-containing cell and non-Nap-containing cells, such as mammalian cells.

In methods, systems, and related antimicrobials and compositions herein described, chlorate can be used as a prodrug against Nar-and/or Nap-containing pathogens and in particular antibiotic-tolerant Nar and/or Nap-containing pathogen and related infections in an individual, in concentrations below the level of toxicity for the individual.

In some embodiments of the methods, systems, and related antimicrobials and compositions, chlorate can be combined with one or more antibiotics to target distinct populations within metabolically stratified aggregate biofilms, where the one or more antibiotics kill cells on the oxic periphery, whereas chlorate kills hypoxic and anoxic cells in the interior. In those embodiments,

In particular in embodiments of the methods, systems, and related antimicrobials and compositions wherein the targeted Nar and/or Nap containing bacteria is within a biofilm a synergic effect with respect to viability of the targeted Nar and/or Nap containing bacteria between the chlorate and the antibiotics has been surprisingly detected as will be understood by skilled person upon reading of the disclosure

The methods, systems, and related antimicrobials and compositions herein described can be used in connection with applications wherein reduction of viability of bacteria and/or reduction of antibiotic resistance is desired, can be used in drug research and to develop diagnostic and therapeutic approaches and tools to counteract infections, in particular Gram negative infections. Additional exemplary applications include uses of the methods, systems, and related antimicrobials and compositions herein described in several fields including basic biology research, applied biology, bio-engineering, biological analysis, aetiology, medical research, medical diagnostics, therapeutics, with particular reference to clinical applications and in additional fields identifiable by a skilled person upon reading of the present disclosure.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and examples sections, serve to explain the principles and implementations of the disclosure.

FIG. 1 shows a diagram illustrating the viable-cell plate counts from P. aeruginosa cultures that were incubated for 4 hr without (untreated) or with 40 μg/ml tobramycin, 10 mM chlorate, 40 μg/ml tobramycin plus 10 mM chlorate, or 10 mM chlorite. Cultures were incubated with these compounds under oxic conditions (black), anoxic conditions with 40 mM nitrate (dark gray), or anoxic conditions without nitrate (light gray). Data show the means of results of 9 biological replicates from 3 independent experiments, and error bars indicate standard errors. This figure demonstrates that chlorate kills oxidant-starved P. aeruginosa cells displaying physiological tolerance to tobramycin.

FIG. 2 shows a diagram illustrating viable-cell plate counts from P. aeruginosa cultures incubated for 4 hr without (untreated) or with 40 μg/ml tobramycin, 10 mM chlorate, 40 μg/ml tobramycin plus 10 mM chlorate, or 10 mM chlorite. Cultures were incubated with these compounds under anoxic conditions with 400 μM nitrate or under anoxic conditions without nitrate. Note that data for anoxic conditions without nitrate are the same as those in FIG. 1 and included for ease of comparison. Data for anoxic conditions with 400 μM nitrate show the means from 3 biological replicates, and error bars indicate standard errors.

FIG. 3 shows diagrams illustrating viable-cell plate counts from P. aeruginosa cultures (top) as well as chlorate concentrations (bottom) over time. P. aeruginosa cultures were incubated without (untreated) or with 1 mM chlorate (chlorate treated) under oxic conditions (A) or under anoxic conditions with 40 mM nitrate (B). Cultures were monitored for 72 hr to determine viable-cell counts (top) and chlorate and nitrate concentrations (bottom) over time. The dashed line in panel B shows the time when nitrate concentrations approximate zero in chlorate-treated cultures. Data show the means of results from 3 biological replicates, and error bars indicate standard errors. In some cases, error bars are smaller than the size of the symbols. This figure demonstrates that chlorate consumption is correlated with cell death during oxidant starvation.

FIG. 4 shows a diagram illustrating nitrate and chlorate concentrations over time. One millimolar chlorate concentrations were monitored in LB that was incubated for 72 hr under oxic conditions (filled circles), anoxic conditions (open circles), and anoxic conditions with 40 mM nitrate (open circles). Forty millimolar nitrate concentrations were monitored in LB medium that was incubated for 72 hr under anoxic conditions plus 1 mM chlorate (filled circles). Concentrations were monitored over time to show that they are stable in the absence of cells. Data show the means from three biological replicates, and error bars indicate standard errors. This figure demonstrates that nitrate and chlorate concentrations are stable in cell-free growth medium.

FIG. 5 shows diagrams illustrating the survival rate (A) and the percentage of chlorate remaining for various WT, narG mutant and napA mutant strains. Strains were grown aerobically, resuspended in fresh medium containing or lacking 1 mM chlorate, and incubated in an anaerobic glove box for 72 hr. (A) The percent survival was calculated for each strain as the viable-cell counts in chlorate-treated cultures divided by the mean viable-cell count in untreated cultures at the end of the incubation, multiplied by 100. (B) The percentage of chlorate remaining was calculated for each strain as the concentration of chlorate in each culture at the end of the incubation divided by the initial concentration in the medium, multiplied by 100. Strains with pEV, pnar, and pnap, indicate strains that contain an empty vector, a vector with narGHJI, and a vector with napEFDABC, respectively. Three biological replicates of both treated and untreated cultures were used in this experiment, and error bars indicate standard errors. This figure demonstrates that nar genes are required for chlorate reduction and sensitivity.

FIG. 6 shows diagrams illustrating cell density (OD500, filled circles) and nitrate concentrations (open circles) monitored over time in the WT and ΔlasR mutant cultures growing under oxic conditions (A), oxic conditions with 40 mM nitrate (B), and anoxic conditions with 40 mM nitrate (C). (D) The WT and ΔlasR cultures were incubated without (squares) or with (filled circles) 1 mM chlorate under anoxic conditions for 72 hr, over which time viable-cell counts (solid lines) and chlorate concentrations (dashed lines) were monitored. Data from all experiments show the means of results from three biological replicates, and error bars indicate standard errors. This figure demonstrates that the ΔlasR mutant has increased rates of nitrate respiration and chlorate consumption and is more sensitive to chlorate.

FIG. 7 shows diagrams illustrating the survival rate and the percentage of chlorate remaining for WT, ΔnarGHJI, ΔlasR and ΔlasRΔnarGHJI. Strains were grown aerobically, resuspended in fresh medium containing or lacking 1 mM chlorate, and incubated in an anaerobic glove box for 72 hr. (A) The percent survival was calculated for each strain as the viable-cell counts in chlorate-treated cultures divided by the mean viable-cell count in untreated cultures at the end of the incubation, multiplied by 100. (B) The percent chlorate remaining was calculated for each strain as the concentration of chlorate in each culture at the end of the incubation divided by the initial concentration in the medium, multiplied by 100. Three biological replicates of both treated and untreated cultures were used in this experiment, and error bars indicate standard errors. This figure demonstrates that nar genes are required for chlorate reduction and sensitivity in the ΔlasR mutant.

FIG. 8 shows a diagram illustrating the percentage of nitrate remaining for WT, ΔlasR mutant and ΔlasRΔnarGHJI mutant. Strains were grown aerobically in LB with 40 mM nitrate for 24 hr, after which the percentage of nitrate remaining in the culture was determined (the final concentration divided by the initial nitrate concentration, multiplied by 100). Data show the means from three biological replicates, and error bars indicate standard errors. This figure demonstrates that increased nitrate utilization in the ΔlasR mutant requires nar genes.

FIG. 9 shows diagrams illustrating: (A) Cartoon of the agar block biofilm assay (ABBA), where cells suspended in agar medium grow as aggregate biofilms. At early incubations, aggregates are uniform in size, but oxygen gradients develop over time, both within the aggregate population and within individual aggregates, leading to a metabolically heterogeneous population. After 12 hr of growth, aggregates are incubated with drugs for 6 hr before they are stained and imaged via confocal microscopy; (B) Representative images of untreated aggregates and those treated with 40 μg/ml tobramycin, 10 mM chlorate, or 40 μg/ml tobramycin plus 10 mM chlorate (combined) are shown at three depths, where cells are stained with SYTO 9 (white; live) and propidium iodide (dark gray; dead). The scale bar is 50 μm for all images; (C) Confocal images were used to generate a sensitivity profile for each treatment condition, where the proportion of dead cells (propidium iodide intensity divided by the sum of the propidium iodide and SYTO 9 intensities) was determined at each depth. The dashed arrow highlights a shift in the depth, where 50% of cells are killed by chlorate in chlorate-only samples and compared to combined-treatment samples. Data show the means from 6 independent experiments, and error bars indicate standard errors. This figure demonstrates that tobramycin and chlorate target distinct populations in aggregate biofilms.

FIG. 10 shows a diagram illustrating oxygen profiles in treated aggregate biofilm populations. Oxygen profiles of P. aeruginosa aggregate biofilms after a 6-hr incubation without a treatment (solid line) or with 40 μg/ml tobramycin (long dashed line), 10 mM chlorate (short dashed line), or 40 μg/ml tobramycin plus 10 mM chlorate (combined; dotted line). The yellow region highlights measurements taken above the surface of the agar, and the dashed black line shows oxygen profiles from uninoculated agar samples. Data show the means from 3 independent experiments, and colored regions indicate standard errors.

### DETAILED DESCRIPTION

Provided herein are methods, systems, and related antimicrobials and compositions that can be used alone or in combination with one or more antibiotics to interfere with viability of bacteria in a medium.

The term “interfere” as used herein in connection with a reference relates to a process or activity resulting in a decrease relative to the reference item compared with a baseline level. Accordingly, interference with reference to viability of bacteria refers to a decrease in viability with respect to a baseline detectable with methods to evaluate viability in a certain medium.

The term “viability” as used here in refers to whether or not a bacterial cell is able to maintain itself or recover its potentiality. Viable cells in the sense of the present disclosure are cells able to, or capable of recover the ability to, form colonies and biofilms on or in a solid or liquid medium. Methods for evaluating the viability of bacteria before and/or after the use of the methods and systems for interference with viability of bacteria described herein include measurement of colony forming units, cell counts such as that described by Wang et al. (J. Bacteriol. 2010, 192, 365-369), and other methods identifiable to a skilled person upon the reading of the present disclosure.

The term “bacteria” or “bacterial cell”, used herein interchangeably with the term “cell” indicates a large domain of prokaryotic microorganisms. Typically, a few micrometers in length, bacteria have a number of shapes, ranging from spheres to rods and spirals, and are present in several habitats, such as soil, water, acidic hot springs, radioactive waste, the deep portions of Earth's crust, as well as in symbiotic and parasitic relationships with plants and animals. Bacteria in the sense of the disclosure refers to several prokaryotic microbial species which comprise Gram-negative bacteria Gram-positive bacteria, Proteobacteria, Cyanobacteria, Spirochetes and related species, Planctomyces, Bacteroides, Flavobacteria, Chlamydia, Green sulfur bacteria, Green non-sulfur bacteria including anaerobic phototrophs, Radioresistant micrococci and related species, Thermotoga and Thermosipho thermophiles as would be understood by a skilled person.

More specifically, the wording “Gram positive bacteria” refers to bacteria that are stained dark blue or violet by Gram staining comprising cocci, nonsporulating rods and sporulating rods, such as, for example, Actinomyces, Bacillus, Clostridium, Corynebacterium, Erysipelothrix, Lactobacillus, Listeria, Mycobacterium, Myxococcus, Nocardia, Staphylococcus, Streptococcus, Streptomyces and additional bacteria identifiable by a skilled person. The wording “Gram-negative bacteria” refers to bacteria that do not retain crystal violet dye in the Gram staining protocol.comprising Escherichia coli (E. coli), Salmonella, Shigella, and other Enterobacteriaceae, Pseudomonas, Moraxella, Helicobacter, Stenotrophomonas, Bdellovibrio, acetic acid bacteria, Legionella, cyanobacteria, spirochaetes, green sulfur, and green non-sulfur bacteria and additional bacteria identifiable by a skilled person.

Bacteria in the sense of the disclosure comprise persister cells which typically constitute a small portion of a culture which is tolerant to killing by lethal doses of bactericidal antibiotics. Persister bacterial cells can be identified, for example, by exposure of logarithmic or stationary cultures of the bacteria to antibiotics using concentrations exceeding five times the minimum inhibitory concentration for each antibiotic. Persister numbers can be determined by plating the antibiotic-treated cultures on LB agar plates and subsequent counting of colony forming units representing the cell numbers which survived antibiotic exposure. Other methods for identification of persister cells will be known by a skilled person, and can be found, for example, in Möker et al. [1].

In embodiments herein described, bacteria in the sense of the disclosure comprise Nar-containing bacteria. “Nar-containing bacteria” refer to the types of bacteria containing a gene set encoding cytoplasmic nitrate reductase (“Nar”), thus capable of conducting Nar-mediated nitrate respiration.

The term “Nar” “nitrate reductase” refers to a group of membrane-bound protein complexes that reduce nitrate to nitrite. Nar is bound to the inner membrane and its active site is located in the cytoplasm. In its reaction, Nar transfers electrons from a membrane-associated reduced quinone to nitrate, thus producing nitrite. This energetically favorable reaction is coupled to proton translocation to generate a proton motive force, which can ultimately be used to power the cell (e.g. ATP synthesis) [2]. Nar is capable of using nitrate as an electron acceptor to reduce nitrate to nitrite during anaerobic respiration. as an alternative to using oxygen as a terminal electron acceptor. The membrane-bound Nar complex is composed of three subunits: a) a catalytic α subunit, encoded by narG, containing a molybdopterin cofactor; b) a soluble β subunit, encoded by narH, containing four [4Fe-4S] centeres; and c) the γ subunit, encoded by narI, containing two b-type hemes. In some embodiments, formation of the Nar complex further requires a chaperone-like component required for the maturation of the αβ complex encoded by narJ gene.

Accordingly, in some embodiments, Nar in the sense of the current disclosure is encoded by a narGHJI operon possessed by the Nar-containing bacteria. narG, H, I encode the α, β, and γ subunit respectively, while narJ encodes the chaperone-like component required for the maturation of the αβ complex. The transcription of narGHJI is typically activated under hypoxic or anoxic conditions and further stimulated by the presence of nitrate.

The term “operon” is a functioning unit of DNA containing a cluster of genes under the control of a single promoter as will be understood by a person of ordinary skill in the art. The term “gene” as used herein indicates a polynucleotide encoding for a protein that in some instances can take the form of a unit of genomic DNA within a bacteria, plant or other organism.

The term “polynucleotide” as used herein indicates an organic polymer composed of two or more monomers including nucleotides, nucleosides or analogs thereof. The term “nucleotide” refers to any of several compounds that consist of a ribose or deoxyribose sugar joined to a purine or pyrimidine base and to a phosphate group and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers respectively to a nucleotide or nucleoside in which one or more individual atoms have been replaced with a different atom or a with a different functional group. Accordingly, the term polynucleotide includes nucleic acids of any length, and in particular DNA RNA analogs and fragments thereof

The term “protein” as used herein indicates a polypeptide with a particular secondary and tertiary structure that can interact with another molecule and in particular, with other biomolecules including other proteins, DNA, RNA, lipids, metabolites, hormones, chemokines, and/or small molecules. The term “polypeptide” as used herein indicates an organic linear, circular, or branched polymer composed of two or more amino acid monomers and/or analogs thereof. The term “polypeptide” includes amino acid polymers of any length including full-length proteins and peptides, as well as analogs and fragments thereof. A polypeptide of three or more amino acids is also called a protein oligomer, peptide, or oligopeptide. In particular, the terms “peptide” and “oligopeptide” usually indicate a polypeptide with less than 100 amino acid monomers. A protein “sequence” indicates the order of the amino acids that form the primary structure

As used herein the term “amino acid”, “amino acid monomer”, or “amino acid residue” refers to organic compounds composed of amine and carboxylic acid functional groups, along with a side-chain specific to each amino acid. In particular, alpha- or α-amino acid refers to organic compounds composed of amine (—NH2) and carboxylic acid (—COOH), and a side-chain specific to each amino acid connected to an alpha carbon. Different amino acids have different side chains and have distinctive characteristics, such as charge, polarity, aromaticity, reduction potential, hydrophobicity, and pKa. Amino acids can be covalently linked to form a polymer through peptide bonds by reactions between the amine group of a first amino acid and the carboxylic acid group of a second amino acid. Amino acid in the sense of the disclosure refers to any of the twenty naturally occurring amino acids, non-natural amino acids, and includes both D an L optical isomers.

Identification of a Nar-containing bacterium can be performed by various techniques. In some embodiments, Nar-containing bacteria can be identified by performing a database search using narG gene or amino acid sequence from a characterized Nar as a query sequence or reference sequence. Bacteria containing a gene or protein sequence having protein having at least 80% query coverage and at least 50% sequence similarity with respect to the reference sequence are identified as Nar-containing bacteria.

As used herein, “query coverage” refers to the percentage of the query sequence that overlaps the identified sequence. The term “sequence similarity” refers to a quantitative measurement of the similarity between sequences of a polypeptide or a polynucleotide. In particular, “sequence similarity” makes reference to the nucleotide bases or protein residues in the two sequences that are the same when aligned for maximum correspondence over a specified comparison window. When percentage of a sequence similarity is used in reference to proteins, it is recognized that residue position which are not identical often differ by conservative amino acids substitutions, where amino acid residues are substituted with a functionally equivalent residue of the amino acid residues with similar physiochemical properties and therefore do not change the functional properties of the molecule. Accordingly, similarity between two sequences can be expressed as percent sequence identity and/or percent positive substitutions. Widely used similarity searching programs, like BLAST, PSI-BLAST [3], SSEARCH [4] [5], FASTA [6] and the HMMER3 [7] programs produce accurate statistical estimates, ensuring protein sequences that share significant similarity also have similar structures.

A functionally equivalent residue of an amino acid used herein typically refers to other amino acid residues having physiochemical and stereochemical characteristics substantially similar to the original amino acid. The physiochemical characteristics include water solubility (hydrophobicity or hydrophilicity), dielectric and electrochemical properties, physiological pH, partial charge of side chains (positive, negative or neutral) and other properties identifiable to a person skilled in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

The similarity between sequences is typically measured by a process that comprises the steps of aligning the two polypeptide or polynucleotide sequences (a subject sequence and a reference sequence) to form aligned sequences, then detecting the number of matched characters in the subject sequence with respect to the reference sequence, i.e. characters similar or identical between the two aligned sequences, and calculating the total number of matched characters divided by the total number of aligned characters in each polypeptide or polynucleotide sequence, including gaps. The similarity result is expressed as a percentage of similarity.

As used herein, “reference sequence” is a defined sequence used as a basis for sequence comparison. A reference sequence may be a subset or the entirety of a specified sequence; for example, as a segment of a full-length protein or protein fragment. A reference sequence can comprise, for example, a sequence identifiable in a database such as GenBank and UniProt and others identifiable to those skilled in the art.

As understood by those skilled in the art, determination of percentage of similarity between any two sequences can be accomplished using a mathematical algorithm. Non-limiting examples of such mathematical algorithms are the algorithm of Myers and Miller [8], the local homology algorithm of Smith et al. [9]; the homology alignment algorithm of Needleman and Wunsch [10]; the search-for-similarity-method of Pearson and Lipman [11]; the algorithm of Karlin and Altschul [12], modified as in Karlin and Altschul [13]. Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA [11], and TFASTA in the Wisconsin Genetics Software Package, Version 8 (available from Genetics Computer Group (GCG), 575 Science Drive, Madison, Wis., USA). Alignments using these programs can be performed using the default parameters.

In some embodiments, identification of a Nar-containing bacterium can be performed by performing a database search using narG amino acid sequence from P. aeruginosa NarG having sequence:

(SEQ ID NO 1)
MSHLLDRLQFFKKKQGEFADGHGETSNESRAWEGAYRQRWQHDKIVRSTH
GVNCTGSCSWKIYVKNGLITWETQQTDYPRTRPDLPNHEPRGCPRGASYS
WYIYSANRLKYPKVRKPLLKLWREARAQHGDPVNAWASIVEDAAKAKSYK
SQRGLGGFVRSSWDEVTEIIAAANVYTAKTYGPDRVIGFSPIPAMSMVSY
AAGARYLSLIGGVCLSFYDWYCDLPPASPQIWGEQTDVPESADWYNSSYI
IAWGSNVPQTRTPDAHFFTEVRYKGTKTVSITPDYSEVAKLTDLWLNPKQ
GTDAALGMAFGHVILKEFHLDRPSAYFVDYCRQYTDMPMLVLLEEHAGGA
FKPTRYLRAADLADNLGQDNNPEWKTIAYDERSGGLVSPTGAIGYRWGES
GKWNIAELDGRSGDQTRLQLSLLDGPEHACEVAFPYFAGQEHPHFKGVAN
DEVLLRRVPFREIVAADGKRLRVATVYDLQMANYSIDRGLGGDNVATSYE
DADTPYTPAWQERITGVPAARATQVAREFADSADKTRGKAMVIIGAAMNH
WYHMDMNYRAVINMLMMCGCIGQSGGGWAHYVGQEKLRPQTGWAPLAFGL
DWSRPPRQMNGTSFFYLHSSQWRHEKLSMHEVLSPLADASRFAEHALDYN
IQAERLGWLPSAPQLNRNPLRIAAEAEAAGLPVADYVVRELKSGGLRFAS
ESPDDPQNFPRNMFIWRSNLLGSSGKGHEYMLKYLLGAKNGVMNDDLGKA
GGPRPTEVDWVDDGAEGKLDLVTTLDFRMSSTCMYSDIVLPTATWYEKDD
LNTSDMHPFIHPLSAATDPAWEAKSDWEIYKAIAKKFSAVAEGHLGVEQD
LVTVPLLHDTPTELAQPFGGDGHDWKKGECEPMPGRNLPTLHLVERDYPN
VYRKFTSLGPLLDKLGNGGKGIGWNTEKEVKLVGDLNHRVVESGVSQGRP
RIDSAIDAAEVVLALAPETNGQVAVKAWEALSKITGREHAHLALPKEDEK
IRFRDIQVQPRKIISSPTWSGLEDEHVSYNAGYTNVHELIPWRTITGRQQ
FYQDHPWMQAFGEGFVSYRPPVNTRTTEKLLNRKPNGNPEITLNWITPHQ
KWGIHSTYSDNLLMLTLSRGGPIIWLSEHDAAKAGIVDNDWVEVFNANGA
ATCRAVVSQRVKDGMVMMYHAQERIVNVPGSETTGTRGGHHNSVTRVVLK
PTHMIGGYAQQAWGFNYYGTVGCNRDEFVVVRKMSKVDWLDEPRHGGLGG
DALPQPLPQDI

as a reference sequence to search for homologs in public databases such as GenBank, UniProt, EMBL, and others identifiable to a person skilled in the art, using tools such as BLASTp and additional tools identifiable by a skilled person. In those embodiments, Nar-containing bacteria can be identified as those containing a protein having at least 80% query coverage and at least 50% sequence similarity compared to SEQ ID NO: 1.

In particular, bacteria containing a gene or protein sequence having protein having at least 80% query coverage and at least 50% sequence similarity with respect to the SEQ ID NO: 1 of P. aeruginosa.

In some embodiments, identification of a Nar-containing bacterium can be performed by isolating cell membrane fractions and performing membrane fraction assay for nitrate reduction by detecting nitrite concentration. In addition, identification of a Nar-containing bacterium can also be performed by constructing a bacterial culture supplemented with chlorate and detecting chlorite concentration after incubation. The procedure can further comprise testing whether the chlorate reduction is inhibited by other compounds such as azide, cyanide, and thiocyanate. [14]

In some embodiments, identification of a Nar-containing bacterium can be performed by culture-independent techniques, such as performing whole genome sequencing and BLAST annotated protein sequences to a P. aeruginosa Nar as described herein. In particular, whole genome sequencing can be performed using culture-dependent methods (e.g. isolate bacterium, culture, extract DNA, sequence) or through culture-independent methods (e.g. single-cell sequencing. Another culture independent technique that can be performed to detect Nar-containing bacteria is sequencing a community's metagenome from an environment, with or without culturing. Metagenomic will provide an indication of whether Nar exists within a community, or with enough depth/coverage allow one to assemble genomes of individuals from the community.

In some embodiments, identifying nar-containing bacterium can be performed by detecting genes encoding Nar. For example, detecting genes encoding Nar can be performed by detecting sequences of one or more of the narG, narH, narJ and narI in the genome, transcriptome, or proteome of one or more candidate bacteria as described above. Exemplary techniques that can be used to detecting sequences of one or more genes (e.g. where the genome is known), comprises computer-based tools for comparing gene sequences, transcript sequences, or protein sequences, such as those using the Basic Local Alignment Search Tool (BLAST) or any other similar methods known to those of ordinary skill in the art.

In some embodiments, detecting genes encoding Nar in the one or more candidate Nar-containing bacteria can be performed by detecting the genes and/or related transcript in the one or more candidate bacteria. Exemplary techniques comprise wet bench approaches such as DNA sequencing, PCR, Southern blotting, DNA microarrays, or other methods of hybridization of DNA or RNA probes to DNA, wherein probes are attached to a label capable of emitting a signal such as radiolabeling, fluorescence, luminescence, mass spectroscopy or colorimetric methods. Exemplary probes that can be used comprise primers from known narG, narH, narJ and narI and/or related transcript as will be understood by a skilled person.

In some embodiments, detecting genes encoding Nar in the one or more candidate bacteria strains can be performed by detecting transcripts of narG, narH, narJ and narI. Exemplary techniques comprise RNA sequencing, PCR, quantitative PCR, Northern blotting, in situ hybridization, RNA microarrays, or other methods of hybridization of DNA or RNA probes to RNA.

In some embodiments, detecting genes encoding Nar in the one or more candidate bacteria strains can be performed by detecting proteins encoded by narG, narH, narJ and narI. Exemplary techniques comprise proteomics, antibody-based methods including immunohistochemistry, immunofluorescence, western blotting, or any other method of protein detection.

In embodiments herein described, the conditions and parameters to use probes/primers to detect narG, narH, narJ and narI can be varied to permit lower or higher threshold or stringency of detection, to ensure hybridization within at least 80% sequence identity at gene level in view of the specific primers/probes selected. For example, use of oligonucleotides comprising one or more degenerated nucleotide bases or using an antibody that binds to more highly conserved protein regions, can require modification of the detection conditions as will be understood by a skilled person.

In an exemplary embodiment, the detection can be done, for example, by isolating genomic DNA from a candidate strain and performing PCR using primer sequences designed to amplify narG gene from known Nar-containing bactera, including the primers listed in the Example section. Alternatively, RNA samples can be isolated from the candidate and these transcripts can be sequenced, and expression of the narG gene can be detected by identification of this gene using homology-based computational identification (e.g. BLAST).

Other methods for identifying a bacterium capable of nitrate respiration would be identifiable to a skilled person upon reading of the present disclosure.

In some embodiments, exemplary Nar-containing bacteria include Pseudomonas aeruginosa, Staphyloccocus aureus, Proteus spp. Escherichia coli, Propionibacterium acnes, Mycobacterium tuberculosis. Exemplary bacteria in the sense of the disclosure can also include Pseudomonas, Actinomyces israelii, Actinomyces gerencseriae, Brevibacterium, Brevibacterium linens, Coryneform Bacteria, Corynebacterium diphtheria, Nocardia, Bacillus anthracis, Bacillus cereus, Brucella melitensis, Brucella suis, Brucella abortus, Burkholderia cenocepacia, Burkholderia pseudomallei, Pantoea agglomerans, Pectobacterium atrosepticum, Propionibacterium propionicus, Pseudomonas fluorescens, Salmonella enterica, Shigella species, Staphylococcus epidermidis, Streptomyces anulatus, and related species that contains Nar to facilitate various physiological functions identifiable to a skilled person upon reading of the present disclosure.

In some embodiments, the Nar-containing bacteria comprise P. aeruginosa, S. aureus, E. coli, wherein the nar operon is expressed under hypoxic/anoxic conditions. In particular, in P. aeruginosa, the presence of nitrate is known to further increase transcription of narGHJI.

In some embodiments where P. aeruginosa is the microorganism, sequences for the genes of the nar operon comprises

P. aeruginosa narG (SEQ ID NO: 2):
ATGAGTCACCTGCTCGACCGCCTGCAGTTCTTCAAGAAGAAGCAGGGCGA
ATTCGCCGATGGCCACGGCGAGACCAGCAACGAGAGCCGCGCCTGGGAAG
GTGCCTACCGGCAGCGCTGGCAGCACGACAAGATCGTGCGCTCCACCCAC
GGGGTGAACTGCACCGGCTCCTGCTCCTGGAAGATCTACGTGAAGAACGG
CCTGATCACCTGGGAAACCCAGCAGACCGACTACCCGCGCACCCGTCCGG
ACCTGCCCAACCACGAGCCGCGCGGCTGCCCGCGCGGGGCCAGCTATTCC
TGGTACATCTACAGCGCCAACCGCCTGAAGTACCCGAAGGTGCGCAAGCC
GTTGCTCAAGCTCTGGCGCGAGGCGCGGGCGCAGCACGGCGACCCGGTGA
ACGCCTGGGCCAGCATCGTCGAGGACGCCGCCAAGGCGAAGAGCTACAAG
AGCCAGCGCGGCCTGGGCGGCTTCGTCCGTTCCAGCTGGGACGAGGTCAC
CGAGATCATCGCCGCGGCCAACGTCTACACCGCCAAGACCTACGGTCCGG
ACCGGGTGATCGGCTTCTCGCCGATCCCGGCCATGTCGATGGTCAGCTAC
GCCGCCGGCGCCCGCTACCTGTCGCTGATCGGCGGGGTCTGCCTGAGCTT
CTACGACTGGTACTGCGACCTGCCGCCGGCCAGCCCGCAGATCTGGGGCG
AGCAGACCGACGTGCCGGAGTCGGCCGACTGGTACAACTCCAGCTACATC
ATCGCCTGGGGCTCCAACGTGCCGCAGACGCGGACCCCGGACGCGCACTT
CTTCACCGAGGTGCGCTACAAGGGCACCAAGACCGTCTCCATCACCCCGG
ACTATTCCGAGGTGGCCAAGCTCACCGACCTCTGGCTCAACCCCAAGCAG
GGCACCGACGCCGCGCTGGGCATGGCCTTCGGTCACGTGATCCTGAAGGA
ATTCCACCTCGACCGGCCGAGCGCCTACTTCGTCGACTACTGCCGCCAGT
ACACCGACATGCCGATGCTGGTGTTGCTGGAAGAACACGCCGGCGGCGCG
TTCAAGCCGACCCGCTACCTGCGCGCCGCCGACCTGGCGGACAACCTCGG
CCAGGACAACAACCCCGAGTGGAAGACCATCGCCTACGACGAGCGCAGCG
GCGGGCTGGTCTCGCCCACCGGCGCCATCGGCTATCGCTGGGGCGAGTCA
GGCAAGTGGAACATCGCCGAGCTGGACGGCAGGAGCGGTGACCAGACGCG
CCTGCAACTGTCGCTGCTCGATGGCCCGGAACATGCCTGCGAGGTGGCCT
TCCCGTATTTCGCCGGGCAGGAGCACCCGCACTTCAAGGGCGTCGCCAAC
GACGAGGTACTGCTGCGCCGGGTGCCGTTCCGCGAGATCGTCGCGGCGGA
CGGCAAGCGCCTGCGGGTGGCCACCGTCTACGACCTGCAGATGGCCAACT
ACAGCATCGACCGCGGCCTGGGCGGCGACAACGTGGCGACCTCCTACGAG
GACGCCGACACGCCCTATACCCCGGCCTGGCAGGAGCGCATCACCGGCGT
TCCGGCGGCGCGCGCGACGCAGGTCGCCCGCGAGTTCGCCGACAGCGCCG
ACAAGACCCGCGGCAAGGCGATGGTGATCATCGGCGCGGCGATGAACCAC
TGGTACCACATGGACATGAACTACCGCGCGGTCATCAACATGCTGATGAT
GTGCGGCTGCATCGGCCAGAGCGGCGGCGGCTGGGCGCACTATGTCGGCC
AGGAGAAGCTGCGCCCGCAGACCGGCTGGGCGCCGCTGGCCTTCGGCCTG
GACTGGAGCCGGCCGCCGCGGCAGATGAACGGCACCAGCTTCTTCTACCT
GCACAGCTCGCAATGGCGCCACGAGAAGCTGTCGATGCACGAGGTGCTGT
CGCCGCTGGCCGACGCCAGCCGCTTCGCCGAACACGCCCTGGACTACAAC
ATCCAGGCCGAACGCCTCGGCTGGCTGCCGTCGGCGCCGCAACTGAACCG
CAACCCGCTGCGCATCGCCGCCGAGGCCGAGGCCGCCGGCCTGCCGGTCG
CCGACTACGTGGTGCGCGAACTGAAGAGCGGCGGCCTGCGCTTCGCCAGC
GAATCGCCGGACGATCCGCAGAACTTCCCGCGCAACATGTTCATCTGGCG
CTCCAACCTGCTGGGCTCCTCCGGCAAGGGCCACGAGTACATGCTCAAGT
ACCTGCTCGGGGCGAAGAACGGGGTGATGAACGATGACCTCGGCAAGGCC
GGCGGTCCGCGTCCCACCGAGGTCGACTGGGTTGACGACGGTGCCGAGGG
CAAGCTCGACCTGGTCACCACCCTGGACTTCCGCATGTCCTCCACCTGCA
TGTACTCGGACATCGTCCTGCCGACCGCTACCTGGTACGAGAAGGACGAC
CTCAACACCTCCGACATGCACCCCTTCATCCATCCGCTGTCGGCGGCCAC
CGATCCGGCCTGGGAAGCCAAGAGCGACTGGGAGATCTACAAGGCCATCG
CCAAGAAGTTCTCCGCCGTCGCCGAAGGCCACCTCGGCGTGGAGCAGGAC
CTGGTCACGGTGCCGCTGCTGCACGACACCCCCACCGAGCTGGCGCAGCC
GTTCGGCGGCGACGGCCATGACTGGAAGAAGGGCGAGTGCGAGCCGATGC
CGGGACGCAACCTGCCGACGCTGCACCTGGTCGAGCGCGACTACCCGAAC
GTCTACCGCAAGTTCACCTCGCTCGGTCCGCTGCTGGACAAGCTGGGCAA
CGGCGGCAAGGGCATCGGCTGGAACACCGAGAAGGAAGTGAAGCTGGTCG
GCGACCTCAACCATCGCGTCGTCGAGAGCGGCGTCAGCCAGGGCCGCCCG
CGCATCGACAGCGCCATCGACGCCGCTGAGGTGGTCCTCGCCCTGGCTCC
GGAAACCAACGGCCAGGTCGCGGTCAAGGCCTGGGAAGCGCTGTCGAAGA
TCACCGGCCGCGAGCATGCCCACCTGGCGCTGCCCAAGGAAGACGAGAAG
ATCCGCTTCCGCGACATCCAGGTGCAGCCGCGCAAGATCATCTCCAGCCC
GACCTGGTCCGGCCTCGAGGACGAGCACGTCAGCTACAACGCCGGCTACA
CCAACGTCCACGAGCTGATCCCGTGGCGCACCATCACCGGTCGCCAGCAG
TTCTACCAGGACCACCCGTGGATGCAGGCGTTCGGCGAAGGCTTCGTCAG
CTACCGGCCGCCGGTCAACACCCGGACCACCGAGAAACTGTTGAACAGGA
AGCCCAACGGCAACCCGGAGATCACCCTGAACTGGATCACCCCGCACCAG
AAATGGGGCATCCACTCCACCTACAGCGACAACCTGCTGATGCTCACCCT
GTCGCGCGGCGGTCCGATCATCTGGCTCAGCGAGCACGACGCGGCCAAGG
CCGGGATCGTCGATAACGACTGGGTCGAGGTGTTCAACGCCAACGGCGCG
GCGACCTGCCGCGCGGTGGTCAGCCAGCGGGTCAAGGACGGCATGGTGAT
GATGTACCACGCCCAGGAACGCATCGTGAACGTACCCGGCAGCGAGACCA
CCGGCACCCGTGGCGGCCACCACAACTCGGTGACCCGCGTGGTGCTCAAG
CCGACCCACATGATCGGCGGCTACGCCCAGCAGGCCTGGGGCTTCAACTA
CTACGGCACGGTCGGCTGCAACCGCGACGAGTTCGTCGTGGTGCGCAAGA
TGAGCAAGGTCGACTGGCTGGACGAACCCCGCCACGGCGGACTCGGCGGC
GACGCCCTGCCGCAACCGCTGCCCCAGGACATTTGA
P. aeruginosa narH (SEQ ID NO: 3):
ATGAAAATTCGTTCGCAAGTCGGCATGGTGCTGAACCTCGACAAGTGCAT
TGGTTGCCACACCTGCTCGATCACCTGCAAGAACGTCTGGACCAGCCGCG
AAGGCATGGAGTACGCCTGGTTCAACAACGTCGAGACCAAGCCCGGGATC
GGCTACCCGAAGGAATGGGAAAACCAGGAGAAGTGGAAGGGCGGCTGGGT
GCGCGCGGCGGACGGTTCGATCCGCCCGCGCATCGGCGGCAAGTTCCGCG
TGCTGGCGAACATCTTCGCCAACCCGGACCTGCCCGAGATCGACGACTAC
TACGAACCGTTCGACTTCGATTACCAGCACCTGCATACCGCGCCCAAGGC
CGAGCACCAGCCGGTGGCGCGCCCGCGCTCGCTGGTCTCCGGGCAGCGCA
TGGAGAAGATCGAGTGGGGCCCGAACTGGGAGGAGATCCTCGGCACCGAG
TTCGCCAAGCGGCGCAAGGACAAGAACTTCGACCAGGTCCAGGCGGACAT
CTACGGTGAGTACGAGAACACCTTCATGATGTACCTGCCGCGCCTCTGCG
AGCACTGCCTGAACCCGGCGTGCGTGGCGTCCTGCCCGAGCGGGGCGATC
TACAAGCGCGAGGAGGACGGCATCGTCCTGATCGACCAGGACAAGTGCCG
CGGCTGGCGGATGTGCATCTCCGGCTGCCCGTACAAGAAGATCTACTTCA
ACTGGAAGAGCGGCAAGTCCGAGAAGTGCATCTTCTGCTACCCGCGCATC
GAGGCCGGCCAGCCCACTGTCTGCTCGGAGACCTGCGTCGGGCGCATCCG
CTACCTCGGCGTGCTGCTCTACGACGCCGACCGCATCCACGAAGTGGCCA
GTTGCGAGAACGAGCGCGAGCTGTACGAGAAGCAACTGGAGATCTTCCTC
GATCCGTTCGACCCGGCGGTGATCGCCCAGGCGCGCAAGGACGGGGTGGC
CGACAGCGTCATCGAGGCGGCGCAGAAGTCGCCGGTGTACAAGCTGGCGA
TGGACTGGAAGCTGGCCCTGCCGCTGCACCCGGAATACCGCACGCTGCCG
ATGGTCTGGTACGTGCCACCGCTGTCGCCGATCCAGAACGCCGCCGCCGA
GGGGCACATCGGCAGCGACGGGGTGATCCCGGACGTGGAGTCGCTGCGCA
TCCCCGTGCAGTACCTGGCCAACCTGCTCACCGCCGGCGACACCGCGCCG
GTGCTGCTGGCGCTCAAGCGCCTGCTGGCGATGCGCGCCTACAAGCGCGC
CGAGCACGTCGAAGGCCGCCAGGACCTGGAGGTGCTGGCCAAGGTCGGGT
TGAGCGTGGAGCAGGTGGAGGAGATGTACCGCTACCTGGCCATCGCCAAC
TACGAGGATCGCTTCGTGATCCCCAGCGCGCACCGCGAGGAAGCGCTTTC
CGATGCCTTCGCCGAGCGTTCCGGCTGCGGCTTCAGCTTCGGCAACGGCT
GTTCCGGCGGCAGCAACTCCGCCGTCAACCTGTTCGGCGGCAAGCCGACC
AACCGCCGCGACGTGATCCAGGTCGTGCAGATCCAGGAGTGA
P. aeruginosa narJ (SEQ ID NO: 4):
ATGAACGATCACAGCCAACTGTTCCGCCTGCTCGCCCTGCTGCTCGACTA
TCCACGCGCCGAGCTGCGCGAGGAGAGCCTCGGCCTGCATGCGCTGATCC
GCACCTGCGAACTGCCGGAAGCGCTGCGCGACGGCCTCGCGGCGCTGCTC
AACGAGCTCTGCCAGGGCGACCTGCTGGACGTCCAGGCGCGCTACGACGG
TCTCTTCGAGCGCGGCCGCTCGGTCTCGCTGCTGCTCTTCGAGCACGTCC
ACGGCGAGAGCCGCGACCGTGGCCAGGCGATGGTCGACCTGCTCGACCGC
TATACCGGGGCCGGCCTGCAGATCGACGTACCGGAGCTGC-CGGACTACC
TGCCGCTGTACCTCGAATACCTGTCGCTGCTGCCGTTCGCGGCGGCCAGC
GAAGGGCTCGCCGAAGTGGCGCACATCCTCGGCCTGCTGGCGCTGCGCCT
GGAGGAACGCGGCAGCGCCTACGCGGCGATTTTCGAGGCGTTGCTGGAAC
TCGGCGGCGAGCGCCCGGACCTCGGCGCGTTGCGTCGCGACCAGGCCCAG
GAACAGCGCGACGACAGCCTGGAGGCCATCGACCGGGCCTGGGAGGAAAC
CCCGGTGAGCTTCACCGACCCTGCCGGCGGTTGCCCGTCGAGCAGCGGCC
GCCGTCCGACGGCGTCCACCGAACAACCATTGCAATGGGTCGCCCAGCCG
GTACCGCAGATGCAGTACCGCGCGGCCCGCGAAGGAGTCTGA
P. aeruginosa narI (SEQ ID NO: 5):
ATGTCGACCAATCTTCTGTTCTTCGGGATCTATCCCTATGTCGCGCTGCT
GATCTGCCTGGTCGGCAGCTGGGCGCGCTTCGACCTCTCGCAGTACACCT
GGAAGGCCGGTTCCAGCCAGATGCTCAGCAAGAAGGGCATGCGGGTATAC
AGCAACCTGTTCCACGTCGGCGTGCTGTTCATCCTCGCCGGCCACTTCGT
CGGCCTGCTGACCCCGGCCTCGGTCTACCACCACCTGATCAGCACCGAGA
ACAAGCAACTGCTGGCGATGGTCTCCGGCGGCTTCTTCGGCGTGCTCTGC
TTCATCGGCCTGAGCGGACTGATCCTGCGCCGCCTGACCGACGCGCGGGT
GCGCGCCACCGGCAACGCCTCTGACCTGATGATCCTGCTGGTGCTCTACG
CCCAGCTGATCCTCGGCCTCTCCACCATCGTCGCCTCGACCCATCACATG
GATGGCTCGGTGATGGTGATGCTCGCCGACTGGGCCCAGGCCATCGTCAC
CCTGCGTCCGCTGGCGGCGGCCGAAGCCATCGCGCCGGTGGGCCTGGTCT
ACAAGCTGCACGTCGGCCTGGGCCTGACCCTGTTCGTGCTGTTCCCCTTC
ACCCGCCTGGTGCACATCGTCAGCGCGCCGGTGTGGTACCTGGGCCGGCG
CTACCAGATCGTGCGTCAGAAACGTCCTGCCTGA

Other NarG sequences similar to P. aeruginosa NarG include NarG from gamma proteobacteria such as E. coli having 98% query coverage and 83% sequence similarity with respect to SEQ ID NO: 1, NarG sequence from the gram positive bacterium S. aureus having 97% query coverage and 67% sequence similarity with respect to SEQ ID NO: 1, and the NarG sequence from the delta proteobacterium Anaeromyxobacter sp. Fw109-5 having 95% query coverage and 61% sequence similarity with respect to SEQ ID NO: 1

In some embodiments, the Nar-containing bacteria can comprise additional genetic features, such as mutations and or other changes which typically affect the rate of nitrate respiration and that in some instances can occur over the course of the bacteria's infection.

For example, in some embodiments, the Nar-containing bacteria herein described can comprise a lasR mutation in which lasR function is defective or lost. lasR is a gene encoding a quorum-sensing regulator, so the loss of this gene has pleiotropic effects [15]. One phenotypic trait of lasR mutants is their decreased rates of oxygen respiration and increased rates of Nar-dependent nitrate respiration [16]. lasR mutants have been isolated from human infections such as bacteremia, pneumonia, chronic wounds, and CF [17] and more resistant to some antibiotics. The prominence of lasR mutants has been documented in CF studies, where they are among the most frequently isolated mutants from CF patients [17] and their presence is associated with worse lung function [18]. lasR mutants are also more resistant to antibiotics commonly used to treat P. aeruginosa infections [15, 16].

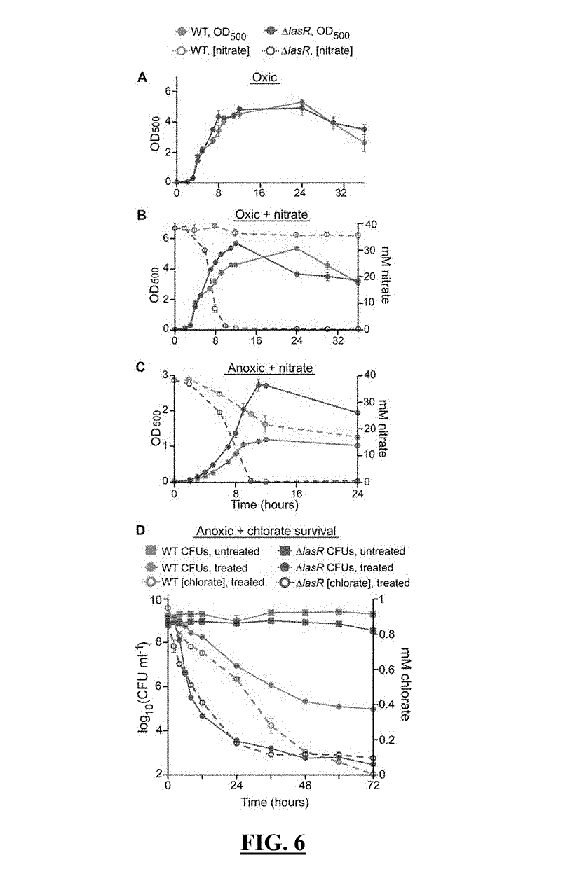

In particular, in some embodiments, Nar-containing bacteria comprising a lasR mutation show increased rates of nitrate respiration and chlorate consumption and reduce chlorate more rapidly than the wild type bacteria does (FIGS. 6-8).

Accordingly, in some of the embodiments herein described, the methods, systems, compounds, and composition herein described are directed to interfere with viability of Nar-containing bacteria comprising a lasR mutation.

In embodiments herein described, methods and systems and related antimicrobials and compositions are used to interfere with viability of Nar-containing bacteria in a medium.

The term “medium” as used herein indicates an environment that is suitable to support growth of microorganisms or cells. In particular, suitable medium comprise growth medium or culture medium in a liquid or gel designed to support the bacteria in vitro, as well as tissues and other suitable environments within a host (including a human host) in vivo. Accordingly, various mediums are formed by or comprise medium components that are chemical compounds and molecules that are used in life-supporting functions and processes of bacteria, which allow bacterial cells to grow and reproduce.

As a person skilled in the art will understand, Nar-containing bacteria are ubiquitous such that many environments support their growth. Accordingly, a medium comprising Nar-containing bacteria can include infection contexts such as any body site in a human or animal having hypoxic/anoxic environments, gastrointestinal tract, particularly distal portions such as ileum and colon, lung sputum, gangrenous tissue, abscesses, ischemic tissue, such as infected skin wounds, organs, such as heart valve (endocarditis), and other hypoxic/anoxic environments identifiable to a person skilled in the art.

A medium in the sense of the disclosure can also include any surfaces or regions that enable the attachment and growth of Nar-containing bacteria such that they can form biofilms. Examples include interior regions where cells experience hypoxia/anoxia, such as piping, such as for water distribution, medical devices such as implants or tubing (e.g. catheters, tracheostomy or feeding tubes) and environmental surfaces such as soil, plants/roots, objects stored in water (e.g. boats).

In some embodiments, the medium can comprise Nap-containing bacteria. “Nap-containing bacteria” refer to the types of bacteria containing a gene set encoding periplasmic nitrate reductase (“NAP”), also capable of reducing chlorate into chlorite. [19]

Nap refers to a group of periplasmic protein complexess that reduce nitrate to nitrite. Nap is encoded by the napEFDABC operon. In the napEFDABC operon, NapAB comprises the periplasmic nitrate reductase, with NapA being the catalytic subunit, with NapC, a cytochrome c-type protein, likely involved in electron transfer to NapAB. NapD is postulated to be a chaperone involved in NapA maturation prior to export into the periplasm. NapF appears to be a non-heme, iron-sulfur protein implicated in an energy conserving role coupled to the oxidation of ubiquinol or play a role in the post-translational maturation of Nap. [20]

In some embodiments, identification of a Nap-containing bacterium can be performed by performing a database search using NapA amino acid sequence from P. aeruginosa NapA having sequence:

(SEQ ID NO: 6)
MNLTRREFAKANAAAIAAAAAGLPILVRASNLVTEADVTSLVWNKAPCRF
CGTGCSVMVATRDGQVVATHGDIKAEVNRGINCVKGYFLSKIMYGSDRLT
RPLLRMKDGKFDKQGEFQPISWEQAFDIMAEKFKAALKAKGPESVGMFGS
GQWTVWEGYAANKLFKAGLRSNNIDPNARHCMASAVMGFMRSFGMDEPMG
CYDDIEATDSFVLWGSNMAEMHPVLWSRVTDRRLSAPQVKVAVLSTFEHR
SFELADLPMVFKPQTDLIILNYIANHIIESGAVNRDFVERHVRFAHGAED
IGYGLRPDDPLEKKAKNADKANTWSDIDFKAFAEFVKPYTLERTARESGV
PAERLKALAELYADPKRKVVSFWTMGFNQHTRGVWANNLIYNIHLLTGKI
SEPGNSPFSLTGQPSACGTAREVGTFSHRLPADLVVTNPKHRETAEKIWK
VPAGTIQEKVGFHAVQQSRMLNDGVLNVYWTQVSNNMQAGPNVMQEVLPG
WRNPDNFVIVSDVYPTVSAQAADLILPSAMWVEKEGAFGNAERRTQFWHQ
LVKAPGEAKSDLWQLVEFSKRFTTDEVWPAELLAKAPELKGKTLYDVLFR
NGQVDRFPASDLAKGYANDEVDAFGFYIQKGLFEEYAAFGRGHGHDLAPF
DAYHEARGLRWPVVDGKETRWRYREGYDPYVSKGSGVQFYGYPDKKAIVF
ALPYEPPAEAPDQDYPFWLATGRVLEHWHTGSMTARVPELYKAVPDALVY
MHPEDARQLKLRRGSEVKVVSRRGEIRARVETRGRNKPPQGLVFVPFFDA
NKLINKVTLDATDPISKQTDYKKCAVRIELLNLA

as a reference sequence to search for homologs in public databases such as GenBank, UniProt, EMBL, and others identifiable to a person skilled in the art, using tools such as BLASTp and additional tools identifiable by a skilled person. In those embodiments, Nap-containing bacteria can be identified as those containing a protein having at least 85% query coverage and at least 55% sequence similarity compared to SEQ ID NO: 6.

In some embodiments, methods herein described comprise contacting an effective amount of chlorate with the Nar-and/or Nap-containing bacteria alone or in combination with an antibiotic and/or other antimicrobial for a time and under conditions to reduce survivability and/or antibiotic resistance of the bacteria.

The term “chlorate” refers to chemical compounds containing chlorate oxyanion having the formula ClO₃⁻

As used herein, “chlorine oxyanion” refers to an anion consisting of one or more oxygen atoms covalently bonded to a chlorine atom. Exemplary chlorine oxyanions include hypochlorite ion ClO⁻, chlorite ion ClO₂⁻, chlorate ion ClO₃⁻, and ClO₄⁻. Chlorine oxyanions are typically comprised within a salt. In particular, a salt of chlorine oxyanion as used herein contains the oxyanion together with a cation as a counterion.

The cation can be a metal cation and in particular the metal ion can have a charge of +1, +2, +3 or +4. Exemplary +1 cation includes Li¹⁺, Na¹⁺, K¹⁺, Cs¹⁺, and Ag¹⁺. Exemplary +2 cation includes Mg²⁺, Ca²⁺, Sr²⁺, Ba²⁺, Ni²⁺, Cu²⁻, Pb²⁺, Fe²⁺ and Zn²⁺. Exemplary +3 cation includes Al³⁺, and Fe³⁺. Exemplary +4 cation includes Ti⁴⁺, Zr⁴⁺.

The cation can be an oxycation which, as used herein, refers to a cation consisting of one or more oxygen atoms covalently bonded to another atom. Exemplary oxycation includes nitronium ion, NO₂¹⁺, and vanadyl ion, VO²⁺.

Exemplary chlorates include potassium chlorate, sodium chlorate, magnesium chlorate, silver chlorate, or in solution as chloric acid. Chlorate can be produced commercially or in laboratory settings. For example, metal chlorates can be prepared by adding chlorine to hot metal hydroxide such as potassium hydroxide or sodium hydroxide as will be understood by a person skilled in the art. The industrial scale synthesis can start from aqueous chloride solution instead of chlorine gas. Chlorate can also be isolated and purified from natural sources as will be understood by a person skilled in the art.

In embodiments of methods herein described, the contacting of chlorate with bacteria is performed for a time and under conditions to reduce antibiotic resistance and/or bacterial survivability, by producing chlorite which is toxic for the cell within the cytoplasm of the cell via Nar-mediated reduction of the chlorate into toxic chlorite. Accordingly, in embodiments herein described the contacting results in inhibition of viability of the Nar-containing bacteria via cytoplasmic chlorite production while minimizing the interference with the viability of possible neighboring cells lacking Nar (see Examples 1-6).

In some embodiments of the methods and systems herein described, chlorate can be administered in an amount between 0.001 mM and 200 mM (see Examples 1-6).

In some of these embodiments, chlorate can be administered in an amount between 0.001 mM and 30 mM.

In some of these embodiments, chlorate can be administered in an amount between 0.001 mM and 10 mM.

In some of these embodiments, chlorate can be administered in an amount between 0.001 mM and 1 mM

In some of these embodiments, chlorate can be administered in an amount between 0.001 mM and 1 mM possibly 0.001 to 0.01 mM

A skilled person will be able to identify a concentration for a particular application in view of the specific medium and specific manner of administration.

In embodiments, wherein selective targeting of Nar and/or Nap containing bacteria is desired the amount of chlorate to be administered can be identified based on the toxicity level of the chlorate in the medium for non-targeted organism possibly present in the medium. In particular in preferred embodiments the amount of chlorate to be administered can be provided selecting a concentration interfering with the viability of the Nar and/or Nap containing bacteria but have an acceptable toxicity level for the non-targeted microorganism.

The term “toxicity” as used herein indicates the degree to which a chemical substance or a particular mixture of substances can damage a target organism. Toxicity can refer to the effect on a whole organism, such as an animal, bacterium, or plant, as well as the effect on a substructure of the organism, such as a cell (cytotoxicity) of an animal or an organ such as the liver (hepatotoxicity) of an animal. The effects of a toxicant are dose-dependent as will be understood by a skilled person and is typically species-specific.

A skilled person can identify the toxicity level of chlorate for non-target organisms present in the medium in view of toxicity guidelines and/or toxicity measurement for the non-target organism in the particular medium in view of the manner of administration.

In preferred embodiments, of methods and systems herein described the chlorate is administered in absence of any one of the other chlorine oxyanion.

As used herein, the term “in the absence” with respect to a chorine oxyanion other than chlorate refers to the presence of, a total amount of chlorine in the form of a chlorine oxyanion other than chlorate in less than 1 ppm by weight, and preferably 0.01 ppm by weight and more preferably 0.001 ppm by weight.

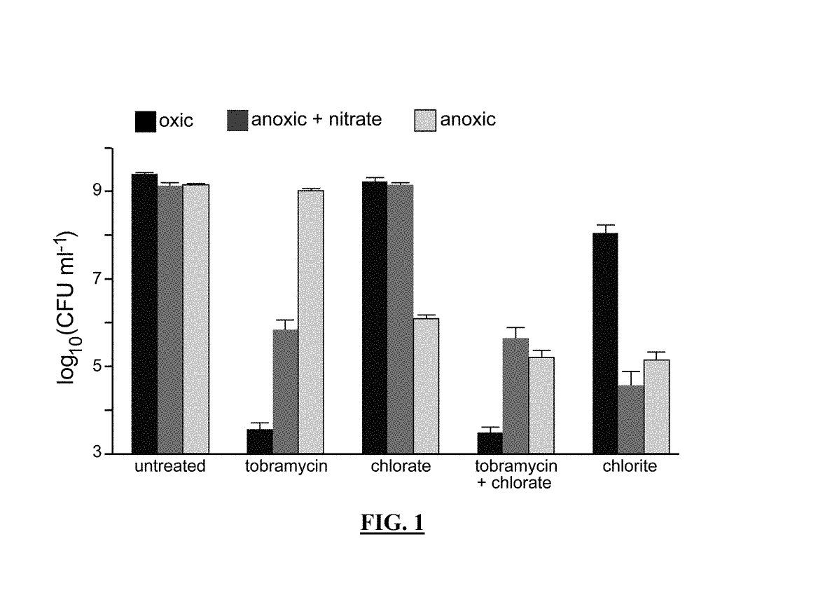

In some embodiments herein described, the composition comprises chlorate in an amount of approximately 10 mM (see Example 1 and FIGS. 1, 2 and 9).

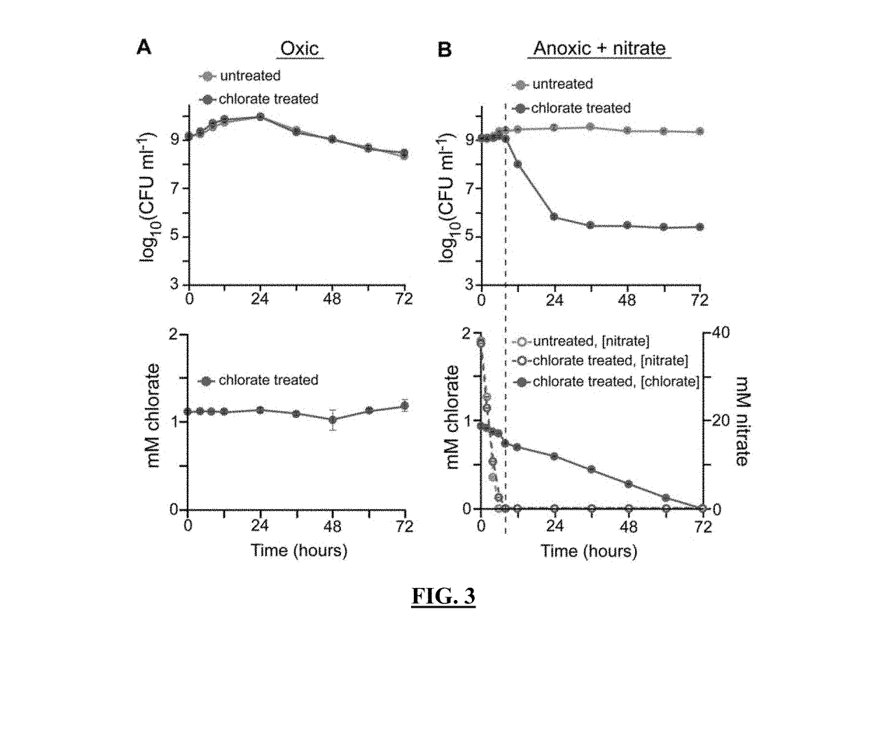

In some embodiments herein described, the composition comprises chlorate in an amount of approximately 1 mM (see FIGS. 3-5 and 7).

In some embodiments, the medium under oxic, hypoxic or anoxic conditions can be further supplemented with nitrate to stimulate the expression of Nar. In these embodiments, nitrate can be supplied to the medium in an amount between 0.1 mM and 50 mM, prior to or simultaneously with the administration of chlorate.

In some embodiments, chlorate and nitration are provided at a concentration ratio of at least 10:1.

In some embodiments, chlorate and nitration are provided at a concentration ratio of at least 4:1.

Chlorate and/or nitrate can be supplied to a medium such as a surface or a host either before the growth of bacteria to inhibit their growth or after bacterial growth has been detected to decrease bacterial viability.

In some embodiments, in addition to nitrate, a carbon source that is known to be metabolized by the bacterium can be provided to the medium. The metabolized carbon source can provide a source of electrons that are required for the reduction of chlorate. Examples of carbon sources that can be provided are sugars such as glucose, peptides or amino acids, glycerol, fatty acids, or carboxylic acids such as acetate, pyruvate, succinate, malate, fumarate, citrate, alpha-ketoglutarate, other types of metabolic intermediates or metabolizable carbon substrates. These carbon sources can be provided to cells at concentrations ranging from 0.1 mM to 50 mM. Similar to nitrate, a carbon source can be provided prior to or simultaneously with the administration of chlorate.

In some embodiments, prior to or concurrently with the administration of chlorate, the medium under oxic conditions can be oxidant-starved to increase Nar expression. The term “oxic” refers to an environment, a condition, or a habitat in which oxygen is present.

In some embodiments, the bacteria in a medium can be oxidant starved by depriving them of an electron acceptor. For example, aerobically respiring bacteria will be oxidant starved when oxygen is removed from the medium while anaerobically respiring bacteria will be oxygen starved when their electron acceptor is removed from the environment. Examples of electron acceptors used for anaerobic respiration include nitrate, sulfate, fumarate, or iron(III). An exemplary method to remove oxygen, comprises flushing the environment with another gas, such as nitrogen or argon. An exemplary method to remove anaerobic electron acceptors, comprises flushing the environment with a liquid medium such as water or phosphate buffer that lacks the electron acceptor, effectively washing it out of the environment.

In some embodiments, the chlorate can be administered together in a combination with nitrate, wherein chlorate is in an amount between 0.001 and 200 mM and nitrate in an amount between 0.1 and 50 mM.

In some embodiments, chlorate can be provided to a medium under hypoxic or anoxic condition. In particular, chlorate can be used to kill oxidant-starved bacteria displaying physiological tolerance to antibiotics. The term “hypoxic” or “anoxic” refers to an environment, a condition, or a habitat, including a host body, deprived of adequate oxygen supply. In such conditions, chlorate can be provided to the medium alone or in combination with one or more antibiotics.

The term “antibiotics” as used herein refers to a type of antimicrobial used in the treatment and prevention of bacterial infection. Some antibiotics can either kill or inhibit the growth of bacteria. Others can be effective against fungi and protozoans. The term “antibiotics” can be used to refer to any substance used against microbes. Antibiotics are commonly classified based on their mechanism of action, chemical structure, or spectrum of activity. Most antibiotics target bacterial functions or growth processes. Antibiotics having bactericidal activities target the bacterial cell wall, such as penicillins and cephalosporins, or target the cell membrane, such as polymyxins, or interfere with essential bacterial enzymes, such as rifamycins, lipiarmycins, quinolones and sulfonamides. Antibiotics having bacteriostatic properties target protein synthesis, such as macrolides, lincosamides and tetracyclines. Antibiotics can be further categoried based on their target specificity. “Narrow-spectrum” antibacterial antibiotics target specific types of bacteria, such as Gram-negative or Gram-positive bacteria. “Broad-spectrum” antibiotics affect a wide range of bacteria.

In some embodiments, suitable antibiotics that can be used in combination with chlorate include ampicillin, kanamycin, ofloxacin, Aminoglycosides, Carbapenems, Ceftazidime, Cefepime, Ceftobiprole, Fluoroquinolones, Piperacillin, Ticarcillin, tobramycin, aztreonam, coliston, tazobactam, and others (or combinations of these antibiotics) that can be readily recognized by a person skilled in the art.

Additional antibiotics that can be used in combination with one or more chlorates herein described include Amoxicillin and clavulanic acid (Augmentin®), Methicillin, oxacillin, nafcillin, cloxacillin, dicloxacillin, cabenicillin, ticarcillin, piperacillin, mezlocillin, azlocillin, ticarcillin and clavulanic acid (Timentin®), piperacillin and tazobactam (Zosyn®), cephalexin, cefdinir, cefprozil, cefaclor, cefuroxime, sulfisoxazole, erythromycin/sulfisoxazole, tobramycin, amikacin, gentamicin, erythromycin, clarithromycin, azithromycin, tetracycline, doxycycline, minocycline, tigecycline, ciprofloxacin, levofloxacin, vancomycin, linezolid, imipenem, meripenem, and aztreonam. As a person of ordinary skill in the art would understand, the antibiotics herein listed can be selected for treating infections or reducing inflammation caused by bacteria including Staphylococcus (S. aureus and S. epidermidis), Pseudomona (P. aeruginosa), Burkholderia cepacian, Escherichia coli, and some mycobacteria.

In some embodiments, suitable antibiotics comprise antibiotics effective against Pseudomonas aeruginosa such as Aminoglycosides, Carbapenems, Ceftazidime, Cefepime, Ceftobiprole, Fluoroquinolones, Piperacillin, Ticarcillin, tobramycin, aztreonam, coliston, and others (alone or in combination) that can be recognized by a skilled person.

The term “antimicrobial” as used herein indicates a substance that kills or inhibits the growth of microorganisms such as bacteria, fungi, or protozoans. Antimicrobial either kills microbes (microbiocidal) or prevent the growth of microbes (microbiostatic).

In some embodiments, chlorate can be administered together with one or more antibiotics either sequentially or in a single administration. Chlorate can be administered to the medium in an amount between 0.001 mM and 200 mM while the antibiotics can be administered according to drug description, FDA guidances, or recommendations by Centers for Disease Control and Prevention (CDC), Infectious Diseases Society of America (IDSA) or other health protection agencies as will be understood by a person skilled in the art.

In some embodiments, the composition comprises chlorate of approximately 10 mM and one or more antibiotics of approximately 40 μg/ml. The one or more antibiotics can comprise tobramycin (see Example 1).

Chlorate can be provided as a solution, cream, gel, or aerosol. A chlorate concentration of between 0.001-200 mM can be applied to the substrate that needs disinfection. Any solution in which chlorate is soluble can be used, such as water. The formula of the provided chlorate can be a salt dissolved in solution (e.g. sodium chlorate) or a dilution of chloric acid. Chlorate can also be incorporated into a polymer such that it can serve to inhibit growth, such as in medical tubing or implant. A surface (e.g. medical tubing or implant) can also be coated with chlorate to inhibit growth.

Chlorate can be provided to an individual via patches, lotion, cream, gel. Chlorate can also be administrated orally, intravenously, intramuscularly, or inhaled as an aerosol or via a nebulizer.

Chlorate dosages can range from 0.001 mM to 200 mM, depending on the administration route. Localized, topical applications can include higher concentrations (e.g. 200 mM) whereas systemic applications can use lower concentrations.

Chlorate and/or nitrate and/or antibiotics can be supplied to a medium such as a surface or a host either before the growth of bacteria to inhibit their growth or after growth has been detected to decrease bacterial viability.

In some embodiments, chlorate is in the form of an aerosol and can thus be delivered topically, e.g. directly into the lungs of an individual and in particular a patient. Methods to deliver chlorate into the lungs of a patient can be identified by a skilled person using, for example, the methods of Corkery (“Inhalable Drugs for Systemic Therapy” Respiratory Care 2000, 45, 931-835) [21].

In some embodiments, the chlorate alone or in combination with one or more antibiotic or antimicrobial is administered to impair bacteria in a medium under hypoxic or anaerobic conditions such as in a biofilm.

As used herein the term “biofilm” indicates an aggregate of microorganisms in which cells adhere to each other on a surface. These adherent cells are frequently embedded within a self-produced matrix of extracellular polymeric substance (EPS). Biofilms can form on living or non-living surfaces and can be prevalent in natural, industrial and hospital settings. The microbial cells growing in a biofilm are physiologically distinct from planktonic cells of the same organism, which, by contrast, are single-cells that can float or swim in a liquid medium. Formation of a biofilm begins with the attachment of free-floating microorganisms to a surface. These first colonists adhere to the surface initially through weak, reversible adhesion via van der Waals forces. If the colonists are not immediately separated from the surface, they can anchor themselves more permanently using cell adhesion structures such as pili. When the biofilm growth is balanced with that of biofilm dispersion, the biofilm is considered “mature.” Methods to quantify and measure biofilms will be known to a skilled person and can include, for example, the COMSTAT method of Heydorn et al. (Microbiology 2000, 146, 2395-2407).

A person skilled in the art would understand that cells within biofilms exhibit physiological heterogeneity partly due to chemical gradients, such as oxygen, existing within these spatially structured communities. As biofilm matures, while the peripheral regions of the biofilm are relatively oxic, cells in deeper layers of the biofilm begin to experience oxygen limitation and redox stress, rendering the cells to be slow growing and highly resistant to antibiotics.

Accordingly, in some embodiments, a method for inhibiting bacteria biofilm formation and/or disrupting mature biofilm in a medium is described, the method comprises contacting chlorate with the medium comprising the biofilm alone or in a combination with one or more antibiotics or antimicrobials. The suitable medium comprises growth medium or culture medium in a liquid or gel designed to support the bacteria in vitro, as well as tissues and other suitable environments within a host (including a human host) in vivo.

In some embodiments, chlorate is combined with one or more antibiotics to target distinct populations within metabolically stratified aggregate biofilms, where the one or more antibiotics kill cells on the oxic periphery, whereas chlorate kills hypoxic and anoxic cells in the interior. In these embodiments, chlorate and antibiotics can act synergistically by targeting distinct bacteria populations, and in particular cells with or without access to oxygen, to substantially prevent and disrupt biofilm aggregate as also exemplified in Examples 5-6.

In some embodiments herein described, the combined treatment of chlorate and antibiotics can be used to disrupt and inhibit pathogenic microbial biofilms in vitro and in vivo.

In particular, in some embodiments, the combination of chlorate and antibiotics can be used to target mature biofilms. Mature biofilms are of significance, for example, because increased resistance to antibiotics (see, for example, Ito et al. Applied and Environmental Microbiology 2009, 75, 4093-4100 and Example 5) [22].

In some embodiments, the chlorate to be used in combination with one or more antibiotics is sodium chlorate. In some embodiments, the one or more antibiotics to be used in combination with chlorate is tobramycin (see Example 5).

In some embodiments, the combined chlorate and antibiotics treatment can be used in vivo in methods and systems for treating and/or preventing a bacterial infection in an individual is described. The method comprises administering to the individual an effective amount of chlorate. In particular, in some embodiments, administering of an effective amount of chlorate can be performed in combination with one or more antibiotics and/or other antimicrobials. In some embodiments, the method is performed to target Nar-containing bacteria. In preferred embodiments, the method is performed by administering to the individual an effective amount of chlorate in absence of other chlorine oxyanions to minimize interference of the treatment with cells other than the targeted bacteria—and in particular to minimize interference of the treatment with viability of the cells of the individual—as will be understood by a skilled person. The system comprises chlorate and an antibiotic and/or other antimicrobial. In some embodiments of methods and systems, the bacteria comprise persister cells.

The term “treatment” as used herein indicates any activity that is part of a medical care for, or deals with, a condition, medically or surgically.

The term “prevention” as used herein indicates any activity which reduces the burden of mortality or morbidity from a condition in an individual. This takes place at primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

The term “condition” as used herein indicates a physical status of the body of an individual (as a whole or as one or more of its parts), that does not conform to a standard physical status associated with a state of complete physical, mental and social well-being for the individual. Conditions herein described include but are not limited to disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms.

The term “individual” as used herein in the context of treatment includes a single biological organism, including but not limited to, animals and in particular higher animals and in particular vertebrates such as mammals and in particular human beings.

In some embodiments, herein described chlorate administration is performed to synergize with biochemical pathways of Nar-containing microorganisms in hypoxic or anoxic host microenvironments during infection. In the absence of oxygen or an alternative electron acceptor, Nar-containing cells become antibiotic tolerant but chlorate sensitive via Nar-dependent reduction. The toxic product, chlorite, from the Nar-dependent chlorate reduction is retained intracellularly thus minimizing the interference with the viability of possible neighboring cells lacking nar such as mammalian cells.

In some embodiments, chlorate is provided at an amount between 0.001 mM and 200 mM. In particular, the amount of chlorate will be selected by the skilled person as not interfering in a deleterious manner with the normal biochemical metabolism of the individual.

In some embodiments, the effective amount of one or more antibiotics can be obtained according to drug description, FDA guidance, or recommendations by Centers for Disease Control and Prevention (CDC), Infectious Diseases Society of America (IDSA) or other health protection agencies as will be understood by a person skilled in the art.

Exemplary antibiotics that can be used in combination with one or more chlorates herein described include Amoxicillin and clavulanic acid (Augmentin®), Methicillin, oxacillin, nafcillin, cloxacillin, dicloxacillin, cabenicillin, ticarcillin, piperacillin, mezlocillin, azlocillin, ticarcillin and clavulanic acid (Timentin®), piperacillin and tazobactam (Zosyn®), cephalexin, cefdinir, cefprozil, cefaclor, cefuroxime, sulfisoxazole, erythromycin/sulfisoxazole, tobramycin, amikacin, gentamicin, erythromycin, clarithromycin, azithromycin, tetracycline, doxycycline, minocycline, tigecycline, ciprofloxacin, levofloxacin, vancomycin, linezolid, imipenem, meripenem, and aztreonam. As a person of ordinary skill in the art would understand, the antibiotics herein listed can be selected for treating infections or reducing inflammation caused by bacteria including Staphylococcus aureus, Pseudomona (P. aeruginosa), Burkholderia cepacian, some mycobacteria.

Suitable dosages can be used which provide the individual with a therapeutically effective amount or a prophylactically effective amount in accordance with the related embodiments of the disclosure. In particular, the term “effective amount” of one or more active ingredients refers to a nontoxic but sufficient amount of one or more drugs to provide the desired effect. For example, an “effective amount” of chlorate associated with the treating and/or preventing (herein also “therapeutically effective amount” or “pharmaceutically effective amount”) a condition in the individual in which bacterial infections are present, refers to a non-toxic but sufficient amount of the chlorate to provide the treatment and/or prevention of such condition in the individual. As another example, an “effective amount” of at least one antibiotic and/or antimicrobial associated with the treating and/or preventing bacterial infection in the individual refers to a non-toxic but sufficient amount of the at least one antibiotic and/or at least one antimicrobial to provide the treatment and/or prevention of the bacterial infection in the individual. A non-toxic amount for chlorate can be identified by a person skilled in the art based on the guidelines and health reference levels provided by health organizations such as WHO and environmental protection agencies such EPA.

As will be readily apparent to one skilled in the art, the useful in vivo dosage to be administered and the particular mode of administration will vary depending upon the age, weight and mammalian species treated, the particular compounds employed, and/or the specific use for which these compounds are employed. The determination of effective dosage levels, that is the dosage levels necessary to achieve the desired result, may be accomplished by one skilled in the art using routine pharmacological methods. Typically, human clinical applications of products are commenced at lower dosage levels, with dosage level being increased until the desired effect and durability of response is achieved (e.g. in clinical practice where a therapeutic effect is sought or dose ranging finding clinical study where selection of a dose associated to a set effect is sought).

Timing and dosages of administration of chlorate alone or in combination with one or more antibiotics and/or antimicrobials to treat and/or prevent bacterial infection herein described can vary depending on the individual treated, the effect to be achieved (treatment and/or prevention) and the severity of the infection as will be understood by a skilled person.

In some embodiments, the composition for treating and/or preventing a bacterial infection in an individual comprises chlorate in an effective amount between 0.001 mM and 200 mM and one or more antibiotics. In some embodiments, the one or more antibiotics comprise tobramycin in an effective amount between 1 mg/kg/day and 10 mg/kg/day.

In some embodiments, the chlorate alone or in combination with one or more antibiotics and/or antimicrobials can be administered once a day, twice a day, three times a day four times a day, or more often as necessary.

In some embodiments, the chlorate alone or in combination with one or more antibiotics and/or antimicrobials can be administered concurrently, combined in a single dosage form. For example, chlorate alone or in combination with one or more antibiotics and/or antimicrobials can be in a single vehicle dissolved in water.

In some embodiments, the chlorate alone or in combination with one or more antibiotics and/or antimicrobials can be administered at the same or at different times in separate dosage forms wherein antibiotic or antimicrobial can be administered before or after chlorate.

In some embodiments, methods herein described chlorate is administered in combination with an antibiotic to individuals in which the antibiotic treatment failed when isolate show in vitro sensitivity to the administered antibiotic. In those embodiments, the chlorate targets oxidant-starved pathogen populations, such as those found in biofilm which are not reached by the antibiotic thus resulting in antibiotic tolerance and treatment failure.

In some embodiments, the methods and systems herein described can be used to treat antibiotic-tolerant infections caused by pathogens known to respire nitrate, such as those that cause infection in the context of chronic wounds, blood infections, skin infections, pneumonia, cystic fibrosis (CF), or those that can inhabit a body site that has hypoxic (micro)environments including abscesses, gangrenous tissue, ischemic tissue, or the gastrointestinal tract. Other infections associated with Nar-containing bacteria include actinomycosis, anthrax, brucellosis, diphtheria, melioidosis, salmonellosis, shigellosis, tuberculosis, urinary tract infections, or gastrointestinal infections.

For example, one of the pathogens known to respire nitrate is Pseudomonas aeruginosa, which causes a range of acute and chronic infections [23]. P. aeruginosa infections contribute to chronic wounds, ventilator-associated pneumonia, and the morbidity and mortality of patients with cystic fibrosis (CF), in whom these infections persist despite aggressive antibiotic treatment [24]. Nitrate respiration can support P. aeruginosa growth and survival in the host, because microenvironments within chronic wounds and the sputum in CF patient lungs contain appreciable anoxic zones and nitrate concentrations [25].

Table 1 below shows a list of exemplary Nar-containing bacterium and their association to bacterial diseases.

TABLE 1

| Nar-containing bacterium | Disease |
|---|---|
| Actinomyces israelii | Actinomycosis |
| Actinomyces gerencseriae | |
| Propionibacterium propionicus | |
| Bacillus anthracis | Anthrax |
| Bacillus cereus | infection |
| Brucella melitensis | Brucellosis |
| B. suis | |
| B. abortus | |
| Corynebacterium diphtheriae | Diphtheria |
| Escherichia coli and other species | various infections |
| Burkholderia pseudomallei | Melioidosis |
| Streptococcus pneumoniae | Pneumococcal infection |
| Salmonella species | Salmonellosis |
| Shigella species | Shigellosis |
| Mycobacterium tuberculosis | Tuberculosis |
| Some Staphylococcus species, | various infections |
| including S. aureus and S. | including chronic |
| epidermidis | wounds |
| Pseudomonas aeruginosa | chronic wounds, ventilator- |
| | associated pneumonia, and |
| | Cystic fibrosis |

In some embodiments, the composition herein described can be used to treat chronic wounds by administering chlorate in combination with one or more antibiotics to substantially disrupt biofilm aggregate and inhibit further biofilm growth.

The term chronic wounds refers to wounds that fail to proceed through the normal phases of wound healing in an orderly and timely manner and often stall in the inflammation phase of healing. Chronic wounds include diabetic foot ulcers, venous leg ulcers, pressure ulcers, and others identifiable to a person skilled in the art.

Exemplary antibiotics that can be used in combination with chlorate and/or nitrate for treating chronic wounds include amoxicillin, clavulanic acid, clindamycin, aminoglycosides, ciprofloxacin, cefalosporines, metronidazole and others identifiable to a person skilled in the art.

Exemplary antimicrobial that can be used in combination with chlorate and/or nitrate for treating chronic wounds include sterile saline or hydrogel, povidone-iodine solutions, cadexomer iodine, hypochlorous acid, collagenase and others identifiable to a person skilled in the art.

In some embodiments, the composition comprises chlorate in an amount ranging between 0.001 mM and 200 mM and one or more antibiotics.

In some embodiments, chlorate is in the form of a patch, lotion, hydrogel, solution or cream and can thus be delivered topically, e.g. directly into the wounds of an individual and in particular a patient. Alternatively, chlorate alone or in combination with the one or more antitiobics or antimicrobial can be provided to an individual intravenously, intramuscularly, or inhaled as an aerosol or via a nebulizer.

Chlorate alone or in combination with the one or more antitiobics or antimicrobial can be given to an individual in the early stages of wound healing (i.e. before a wound is defined as chronic) to inhibit the growth of Nar-containing bacteria or given to an individual after a wound is determined to be chronic to reduce viability of Nar-containing organisms.

In some embodiments, the compositions herein described can be used to treat cystic fibrosis (CF) patient by administering chlorate in combination with one or more antibiotics to substantially prevent and/or disrupt biofilm growth. As used herein, cystic fibrosis is a progressive, genetic disease that causes persistent lung infections and limits the ability to breath over time.

In some embodiments, the composition comprises chlorate in an amount ranging between 0.001 mM and 200 mM.

Exemplary antibiotics suitable in particular for treatment of cystic fibrosis include Amoxicillin and clavulanic acid (Augmentin®), Methicillin, oxacillin, nafcillin, cloxacillin, dicloxacillin, cabenicillin, ticarcillin, piperacillin, mezlocillin, azlocillin, ticarcillin and clavulanic acid (Timentin®), piperacillin and tazobactam (Zosyn®), cephalexin, cefdinir, cefprozil, cefaclor, cefuroxime, sulfisoxazole, erythromycin/sulfisoxazole, tobramycin, amikacin, gentamicin, erythromycin, clarithromycin, azithromycin, tetracycline, doxycycline, minocycline, tigecycline, ciprofloxacin, levofloxacin, vancomycin, linezolid, imipenem, meripenem, and aztreonam. A person skilled in the art would be able to select appropriate antibiotics for treating cystic fibrosis caused by particular pathogen. An exemplary indication of antibiotic, is shown in Table 2 below From Orenstein, D. Cystic Fibrosis: A Guidefor Patient and Family, 4th ed. LWW; 2011.

TABLE 2
An exemplary list of antibiotics for treating CF

| | | |
|---|---|---|
| Type and kinds | Bacteria Treated | How Taken |

Penicillins

| | | |
|---|---|---|
| Amoxicillin and clavulanic | Staphylococcus aureus | |
| acid (Augmentin ®) | (Staph) | |
| Methicillin, oxacillin and | Pseudomonas (P. | Intravenous, |
| nafcillin | aeruginosa) | intramuscular |
| Cloxacillin and dicloxacillin | Staph | Oral |
| Cabenicillin, ticarcillin, | P. aeruginosa | Intravenous |
| piperacillin, mezlocillin and | | |
| azlocillin | | |
| Ticarcillin and clavulanic | Staph, P. aeruginosa | intravenous |
| acid (Timentin ®) | | |
| Piperacillin and tazobactam | P. aeruginosa | intravenous |
| (Zosyn ®) | | |

Cephalosporins

| | | |
|---|---|---|
| Cephalexin, cefdinir, | Staph, P. aeruginosa | oral |
| cefprozil and cefaclor | | |
| Cefuroxime | Staph | oral |

Sulfa

| | | |
|---|---|---|
| Sulfisoxazole | P. aeruginosa | oral |
| Erythromycin/sulfisoxazole | Staph | oral |

Aminoglycosides

| | | |
|---|---|---|
| Tobramycin, amikacin, | P. aeruginosa (in | Intravenous, |
| gentamicin | combination with | inhaled |
| | gentamicin, tobramycin, | |
| | and amikacin; also work | |
| | well with anti- | |
| | Pseudomonas penicillin | |
| | drug) | |

Macrolides

| | | |
|---|---|---|
| Erythromycin, clarithromycin | Staph and may help | Oral, |
| and azithromycin | reduce inflammation | intravenous |
| | from P. aeruginosa | |

Tetracyclines

| | | |
|---|---|---|
| Tetracycline, doxycycline, | Formerly P. aeruginosa, | Oral, |
| minocycline, and tigecycline | some Burkholderia | intravenous, |
| | cepacia and Staph | intramuscular |

Quinolones

| | | |
|---|---|---|
| Ciprofloxacin, levofloxacin | Pseudomonas | Oral, |
| | | intravenous |

Vancomycin

| | | |
|---|---|---|
| Vancomycin | Staph and methicillin- | intravenous |
| | resistant Staphylococcus | |
| | aureus (MRSA) | |

Linezolid

| | | |
|---|---|---|
| Linezolid | MRSA and some | Oral, |
| | mycobacteria | intravenous |

Imipenem & Meripenem

| | | |
|---|---|---|
| Imipenem & Meripenem | P. aeruginosa, Staph | intravenous |

Aztreonam (Cayston ®)

| | | |
|---|---|---|
| Aztreonam (Cayston ®) | P. aeruginosa | Intravenous, |
| | | inhaled |

In some embodiments, chlorate is in the form of an aerosol or mist and can thus be delivered topically, e.g. directly into the lungs of an individual and in particular a patient. Methods to deliver chlorate into the lungs of a patient can be identified by a skilled person using, for example, the methods of Corkery (“Inhalable Drugs for Systemic Therapy” Respiratory Care 2000, 45, 931-835) [21].

Chlorate alone or in combination with one or more antibiotics can also be administered to an individual orally, intravenously or intramuscularly via patches, lotions, creams or other formulations identifiable to a person skilled in the art.

In general, the administering to the individual the chlorate alone or in combination with an antibiotic and/or other antimicrobial can be performed through various administration routes including oral ingestion, inhalation, intranasal, topical application, intravenous or subcutaneous injections and others as will be recognized by a person skilled in the art. Chlorate alone or in combination with an antibiotic and/or other antimicrobial can be in a form of an aqueous solution, cream, solid powder, tablets, aerosols, or other forms as will be understood by a person skilled in the art.

In some embodiments of the methods and systems, the Nar and/or Nap containing bacteria comprise persister cells.

In some embodiments, one or more chlorate salts herein described can be comprised in a composition together with a suitable vehicle. The term “vehicle” as used herein indicates any of various media acting usually as solvents, carriers, binders or diluents for the chlorates that are comprised in the composition as an active ingredient. In particular, the composition including the one or more chlorate salts can be used in one of the methods or systems herein described.

In embodiments, wherein compositions are formulated for administration to an in vitro medium, the composition can comprise one or more chlorate salts are formulated for treatment of medium, the related formulation can comprise the one or more chlorate salts in a carrier solution that can be a buffer, preferably an inorganic buffer such as carbonate or phosphate-based buffer, that has a pH value that allows the one or more chlorate salts to treat medium.

In some embodiments, wherein compositions are formulated for administration to an in vitro medium, the composition for treatment of medium comprises at least one chlorate salt further comprising at least one antibiotics selected from the group consisting of rifamycin class antibiotics, aminoglycoside, amphenicol, ansamycin, beta-Lactam, carbapenem, cephamycin, monobactam, oxacephem, lincosamide, macrolide, polypeptide, tetracycline, a 2,4-diaminopyrimidine class antibiotic, penicillin, neomycin, metronidazole, vancomycin, paromomycin, timidazole, clarithromycin, amoxicillin, sulfasalazine; olsalazie; mesalamine; prednisone; azathioprine; mercaptopurine; methotrexate, ampicillin, clindamycin, rifampicin, rifamycin, vancomycin, chloramphenicol, spectinomycin, fluoroquinolones, and cephalosporins, and combinations thereof. In some embodiments, molar ratio of the at least one antibiotics to the chlorate is 0.1 to 10, preferably 0.5 to 2.

In some embodiments, wherein compositions are formulated for administration to an in vitro medium, a method for one or more chlorate salts having a target bacterium comprises providing a composition having a water-based carrier vehicle, preferably an aqueous buffer at a suitable pH, preferably in a range between pH 4-10, wherein the one or more chlorate salts would be solubilized. The composition of one or more chlorate salts solution would then be sprayed on an environment outside of animal body and as the one or more chlorate salts solution comes into contact with the target bacterium environment outside of animal body, it will reduce or cause to reduce the detectable reduction or elimination of the target bacterium.

In embodiments of methods for decontamination of surfaces, equipment or other environments outside an organism herein described, the rate of the reduction or elimination of the target bacterium is dependent on the temperature of the environment in which the target bacterium is present. For example, the rate of rate of the reduction or elimination of the target bacterium will be higher in summer of 20-40 ° C. than in 4-15 ° C. in the winter.

In some embodiments, wherein compositions are formulated for administration to an in vitro medium, the compositions for treatment of medium, is adjusted according the specific Nar and/or Nap containing bacterium. In some situations, an additional ingredient such as diethylene glycol can be added to compensate extreme environmental conditional to avoid freezing or solubilization of antibiotics. The diethylene glycol can be present in 1 to 50% by weight, preferably 5 to 10% by weight.

In some embodiments, the vehicle is a pharmaceutically acceptable vehicle and the composition is a pharmaceutical composition formulated for administration to an individual. In particular, in some embodiments, disclosed are pharmaceutical compositions which contain at least one or more chlorate salts as herein described, in combination with one or more compatible and pharmaceutically acceptable vehicles

As used herein, the term “pharmaceutically acceptable” means not biologically or otherwise undesirable, in that it can be administered to a subject without excessive toxicity, irritation, or allergic response, and does not cause unacceptable biological effects or interact in a deleterious manner with any of the other components of the composition in which it is contained.

The pharmaceutical preparations of at least one chlorate salt can be given by forms suitable for each administration route. For example, these preparations are administered in tablets or capsule form, by injection, inhalation, eye lotion, eye drops, ointment, suppository, and additional forms identifiable by skilled person, administration by injection, infusion or inhalation; topical by lotion or ointment; and rectal by suppositories. The injection can be bolus or can be continuous infusion. Depending on the route of administration, a pharmaceutical preparation of at least one chlorate salt can be coated with or disposed in a selected material to protect it from natural conditions that can detrimentally affect its ability to perform its intended function. A GI specific at least one chlorate salt can be administered alone, or in conjunction with at least one antibiotic as described herein and/or with a pharmaceutically-acceptable carrier. A pharmaceutical preparation of at least one chlorate salt can be administered prior to the administration of the antibiotic, simultaneously with the antibiotic, or after the administration of the antibiotic.

In some embodiments, the one or more chlorate salts herein described to be used in a method herein described can be included in pharmaceutical compositions together with an excipient or diluent.

The term “excipient” as used herein indicates an inactive substance used as a carrier for the active ingredients of a medication. Suitable excipients for the pharmaceutical compositions herein described include any substance that enhances the ability of the body of an individual to absorb one or more chlorate salts herein described or combinations thereof. Suitable excipients also include any substance that can be used to bulk up formulations with the one or more chlorate salts or combinations thereof, to allow for convenient and accurate dosage. In addition to their use in the single-dosage quantity, excipients can be used in the manufacturing process to aid in the handling of the one or more chlorate salts or combinations thereof concerned. Depending on the route of administration, and form of medication, different excipients can be used. Exemplary excipients include, but are not limited to, antiadherents, binders, coatings, disintegrants, fillers, flavors (such as sweeteners) and colors, glidants, lubricants, preservatives, sorbents.

The term “diluent” as used herein indicates a diluting agent which is issued to dilute or carry an active ingredient of a composition. Suitable diluents include any substance that can decrease the viscosity of a medicinal preparation.

In some embodiments, a pharmaceutical composition to treat a condition associated with exposure of an individual to a Nar and/or Nap containing bacteria includes at least one or more chlorate salts in a therapeutically effective amount and a pharmaceutically acceptable vehicle. In some embodiments, 50-1 g of the at least one or more chlorate salts are formulated in a pyrogen-free aqueous solution. In some embodiment, 600 mg of the one or more chlorate salts are dissolved or suspended in 2 mL of sterile, pyrogen-free solution containing 40 mg benzyl alcohol, 22.5 mg glycine, and Water for Injection, the pH is adjusted with hydrochloric acid to a pH range which is 4.0 to 10.0.

In some embodiments, compositions methods and systems herein described can further comprise an antimicrobial. In some embodiments, the antimicrobial is formulated in an antimicrobial composition further comprising a compatible vehicle, which can be a vehicle for effective administrating and/or delivering of the antimicrobial to a medium.

In some embodiments the antimicrobial can be formulated for administration to an individual

In some embodiments the antimicrobial can include a microbiocidal agent and/or microbiostatic agents.

In some embodiments, the antimicrobial comprises chlorate and optionally a compatible vehicle for effective administrating and/or delivering of the one or more agents to an individual.

In some embodiments, antimicrobial is a pharmaceutical composition comprising chlorate for the treatment of cystic fibrosis and a pharmaceutically acceptable vehicle such as an excipient or diluent.

In some embodiments, the antimicrobial or pharmaceutical composition comprising chlorate herein described further comprises antibiotic and/or an additional antimicrobial.

In some embodiments, the composition can comprise medium components such as sodium thioglycolate (HS—CH₂CO₂Na), sodium dithionite, Organic; simple sugars e.g. glucose, acetate or pyruvate; extracts such as peptone, tryptone, yeast extract etc., hydrogen carbonate salts (HCO₃⁻), amino acids, NH₄Cl, (NH₄)₂SO₄, KNO₃, KCI, K₂HPO₄, MgCl₂, MgSO₄, CaCl₂, Ca(HCO₃)₂, FeCl₃, Fe(NH₄)(SO₄)₂, Fe-chelates, CoCl₂, ZnCl₂, Na₂MoO₄, CuCl₂, MnSO₄, NiCl₂, Na₂SeO₄, Na₂WO₄, Na₂VO₄, Vitamins, amino acids, purines, pyrimidines.

In some embodiments, the composition can further comprise a basic binder, and an isocyanate compound and in particular an isocyanate pre-polymer. In some embodiments, the composition can comprise an aqueous dispersion of an acryl-modified polyester resin, a blocked polyisocyanate compound having a nonionic hydrophilic group; and an aqueous dispersion of acrylic-based polymer fine particles.

As described herein, chlorate, nitrate, antibiotics, bacteria, antimicrobial agents and/or compositions herein described can be provided as a part of systems to perform any methods, including any of the assays described herein. The systems can be provided in the form of arrays or kits of parts. An array, sometimes referred to as a “microarray”, can include any one, two or three dimensional arrangement of addressable regions bearing a particular molecule associated to that region. Usually, the characteristic feature size is micrometers.

In a kit of parts, the chlorate, nitrate, bacteria, and/or other reagents to perform the methods herein described can be included in the kit alone or in the combination with of one or more antibiotic and/or antimicrobial agents compositions. In particular in kit of parts for the treatment of an individual the chlorate, nitrate, bacteria, and/or other reagents can be comprised together with the antibiotic and/or antimicrobial formulated for administration to the individual as well as additional components identifiable by a skilled person.

In a kit of parts, the chlorate, nitrate, bacteria, one or more antibiotic antimicrobial agents compositions and/or other reagents to perform the methods herein described can be comprised in the kit independently. In particular, chlorate, nitrate, antibiotics, and/or antimicrobial agent can be included in one or more compositions, and each component can be in a composition together with a suitable vehicle. In some embodiments, a kit can comprise one or more chlorates with medium components within a composition herein described.

Additional components can include labeled molecules and in particular, labeled polynucleotides, labeled antibodies, labels, microfluidic chip, reference standards, and additional components identifiable by a skilled person upon reading of the present disclosure. The terms “label” and “labeled molecule” as used herein as a component of a complex or molecule referring to a molecule capable of detection, including but not limited to radioactive isotopes, fluorophores, chemiluminescent dyes, chromophores, enzymes, enzyme substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting fluorescence in a detectable image. As a consequence, the wording “labeling signal” as used herein indicates the signal emitted from the label that allows detection of the label, including but not limited to radioactivity, fluorescence, chemiluminescence, production of a compound in outcome of an enzymatic reaction and the like.

In some embodiments, detection of a viable bacteria can be carried either via fluorescent based readouts, in which the labeled antibody is labeled with fluorophore, which includes, but not exhaustively, small molecular dyes, protein chromophores, quantum dots, and gold nanoparticles. Additional techniques are identifiable by a skilled person upon reading of the present disclosure and will not be further discussed in detail.

In embodiments herein described, the components of the kit can be provided, with suitable instructions and other necessary reagents, in order to perform the methods here disclosed. The kit will normally contain the compositions in separate containers. Instructions, for example written or audio instructions, on paper or electronic support such as tapes, CD-ROMs, flash drives, or by indication of a Uniform Resource Locator (URL), which contains a pdf copy of the instructions for carrying out the assay, will usually be included in the kit. The kit can also contain, depending on the particular method used, other packaged reagents and materials (i.e. wash buffers and the like).

The methods, herein described, can be performed in vivo and/or in vitro as will be understood by a skilled person.

### EXAMPLES

The related compositions, methods and systems herein disclosed are further illustrated in the following examples, which are provided by way of illustration and are not intended to be limiting. In particular, the following examples illustrate exemplary chlorate, antibiotics, methods and protocols for inhibiting Nar-containing bacteria. A person skilled in the art will appreciate the applicability and the necessary modifications to adapt the features described in detail in the present section, to additional agents and related compositions, methods and systems according to embodiments of the present disclosure. The following materials and methods were used.

### Bacterial Strains and Growth Conditions.

Strains used in this study include P. aeruginosa PA14 (wild type [WT]), isogenic narG and napA transposon insertion mutants [26], and an isogenic, in-frame, markerless lasR deletion mutant [27]. Strains that were constructed for this work are described below. All experiments used Luria-Bertani medium (LB; Difco) as a growth medium, supplemented with KNO₃ as specified. Aerobic liquid cultures were incubated at 37° C. with shaking at 250 rpm. Anaerobic work was conducted in an anaerobic glove box with a 95% N2-5% H2 atmosphere, and anaerobic cultures were incubated at 33° C.

### Bacterial-Survival Assays and Viability Measurements.

In aerobic-survival endpoint assays, overnight aerobic cultures were pelleted, washed twice with and resuspended in LB, and added to a 96-well microtiter plate at high cell density (optical density at 500 nm [OD₅₀₀]˜3). Wells also contained sodium chlorate (final concentration, 10 mM), sodium chlorite (final concentration, 10 mM), tobramycin (final concentration, 40 μg ml⁻¹), or an equal volume (20 μl) of dH₂O in untreated-control wells (200-μl total volume per well). Ninety-six-well plates were incubated at 37° C. with shaking at 250 rpm for 4 hr before viable cells on plates were counted. Anaerobic-survival endpoint assays were performed as described above, except that overnight cultures were grown anaerobically at 33° C. in LB with 40 mM KNO₃, cells were washed and resuspended in either LB or LB with 40 mM KNO₃ under anaerobic conditions, and 96-well plates were incubated anaerobically at 33° C. for 4 hr and then removed from the glove box so that cultures could be serially diluted for viable-cell plate counts.

Aerobic- and anaerobic-survival assays were also conducted over the course of 72 hr. The experimental setup was the same as described above, except that culture volumes were 5 ml (in capped tubes), cultures were treated with 1 mM sodium chlorate, and anaerobic experiments using LB with 40 mM KNO₃ were buffered with 200 mM MOPS (morpholinepropanesulfonic acid) to prevent an increase in culture pH that results from denitrification reactions. Two-hundred-microliter samples were taken over time and used for viable-cell plate counts and to quantify nitrate and chlorate concentrations (described below).

Viable-cell plate counts were performed by serially diluting samples in phosphate-buffered saline (PBS). Dilutions spanning 7 orders of magnitude were plated on LB agar plates as 10-μl drips. Plates were incubated at 37° C. for ˜20 h, and incubation continued at room temperature (72 hr of total incubation) to allow for the growth of slow-growing colonies. Colonies were counted daily, and numbers of CFU per milliliter were calculated at the end of the incubation period. All viable-cell plate counting was carried out under aerobic conditions.

### Nitrate and Chlorate Quantification.

Culture samples were centrifuged at 16,000×g at room temperature for 10 min to pellet cells. Supernatants were diluted 1:10 in dH₂O and added to 0.5-ml vials (Thermo Fisher Scientific catalog numbers 038010 and 038011). Nitrate and chlorate concentrations were quantified using the Dionex ICS 2000 ion chromatography system (Environmental Analysis Center, Caltech). Samples were loaded via a 15-μl sample loop onto an AS-19 separator (2- by 250-mm) column protected by an AG-19 guard (2 by 50 mm), maintained at 30° C. A hydroxide gradient was produced using a potassium hydroxide eluent generator cartridge and pumped at 0.25 ml per minute. The gradient began with a 10 mM hold for 10 min, increased linearly to 58 mM at 25 min, and remained at 58 mM until the end of data acquisition at 32 min. Seven minutes was allowed between analyses to return the column to initial conditions. Anions were detected at neutral pH using an AERS 500 2-mm suppressor (Thermo Fisher Scientific) operated in eluent recycle mode with an applied current of 30 mA, and the conductivity detection cell was maintained at 35° C. A carbonate removal device (CRD 200, 2 mm) was installed between the suppressor eluent out port and the conductivity detector eluent in port. Sodium chlorate and potassium nitrate standards were used to identify sample analytes via retention time and to generate standard curves for quantifying analyte concentrations. Similar methods were used to quantify chlorite concentrations, but chlorite was not detected in culture samples (1 μM detection limit).

Construction of nar and nap Complement Strains and narGHJI Mutant Strains.

To construct complement strains, the narGHJI genes were amplified from P. aeruginosa using primers 5′-CCATACCCGTTTTTTTGGGCTAGCGAATTCGAGCTCAGGAGGAGATCAAGATGAGTC ACC-3′ (SEQ ID NO: 7) and 5′-GCAAATTCTGTTTTATCAGACCGCTTCTGCGTTCTGATTTAAGGTTCAGGCAGGACGT TT-3′ (SEQ ID NO: 8), and the napEFDABC genes were amplified using primers 5′-CCATACCCGTTTTTTTGGGCTAGCGAATTCGAGCTCAGGAGGCTGGGCCAATGAACG AAC-3′ (SEQ ID NO: 9) and 5′-GCAAATTCTGTTTTATCAGACCGCTTCTGCGTTCTGATTTAAGCGCTACCAGCCCTTC AC-3′ (SEQ ID NO: 10). The fragments were each cloned into HindIII-digested pMQ72 (89) downstream of the arabinose-inducible promoter Para via Gibson cloning (NEB number E2611) (90). The resulting plasmids, pMQ72-narGHJI and pMQ72-napEFDABC, and the empty vector were transformed into Escherichia coli DH10B cells. These plasmids and the empty vector were then introduced into P. aeruginosa WT and mutant strains via triparental conjugation, and successful exconjugants were selected by plating cells on VBMM medium (91) supplemented with 50 μg ml⁻¹ gentamicin.

The narGHJI genes were deleted in WT PA14 and the isogenic ΔlasR background. The region upstream of narG was amplified using primers 5′-TAAAACGACGGCCAGTGCCACGTACTGGGTGTTCGCCCTG-3′ (SEQ ID NO: 11) and 5′-CGCGCAGGGTCTTGATCTCCTCACCCGGTC-3′ (SEQ ID NO: 12), and the region downstream of narI was amplified using primers 5′-GGAGATCAAGACCCTGCGCGGCCGGCGCAT-3′ (SEQ ID NO: 13) and 5′-CATGATTACGAATTCGAGCTGCTGGCGCGGCAGGAAGCGC-3′ (SEQ ID NO: 14). These fragments were cloned into HindIII- and SacI-digested pMQ30 (89) via Gibson cloning. The resulting plasmid was transformed into E. coli DH10B and introduced into P. aeruginosa strains via triparental conjugation, and merodiploids were selected by plating them on VBMM medium (91) supplemented with 50 μg ml⁻¹ gentamicin. ΔnarGHJI mutants were generated by resuspending merodiploid cells in PBS and plating them on LB supplemented with 10% sucrose. Correct mutants were confirmed both via PCR and their inability to grow anaerobically with nitrate.

### Anaerobic Adaptation and Survival.

Overnight cultures were grown aerobically in LB with 40 mM KNO₃, 100 mM L-arabinose, 50 μg ml⁻¹ gentamicin (for strains containing pMQ72-derived plasmids), and 50 μg ml⁻¹ kanamycin (for transposon insertion strains). Overnight cultures were pelleted, washed twice in LB with 100 mM _{L}-arabinose to remove antibiotics, and resuspended in LB with 100 mM _{L}-arabinose with or without 1 mM sodium chlorate at high cell density (OD₅₀₀˜3). Two hundred microliters of resuspended culture was added to 96-well microtiter plates, and the plates were moved to the anaerobic glove box for cells to adapt to the anaerobic lifestyle. After a 72-hr incubation at 33° C., 96-well plates were removed from the anaerobic glove box and viable cells on plates were counted to calculate numbers of CFU per milliliter (described above). Percent survival was calculated for each strain by dividing CFU-permilliliter values from cultures containing chlorate by the average CFU-per-milliliter value from control cultures lacking chlorate and multiplying by 100. CFU-per-milliliter values of untreated control cultures were similar across strains, ranging from 1.0×10⁹ to 2.2×10⁹. The percentage of chlorate remaining after the 72-hr incubation was determined for each strain by dividing the final chlorate concentration in each culture by the initial chlorate concentration in the medium and multiplying by 100.

### Agar Block Biofilm Assays (ABBA).

Overnight aerobic cultures were diluted to an OD₅₀₀ of 0.001 in molten agar growth medium that had been cooled to 44° C. For all agar block biofilm assays (ABBAs), the growth medium used was LB with 5 mM KNO3 and 0.5% noble agar. A portion of the dilution (175 p.1) was added to chambered cover glass slides (Thermo Fisher Scientific number 155409), solidified at room temperature, and incubated at 37° C. for 12 hr in a humidified chamber. After incubation, cells that were not suspended in the agar were removed by adding 400 μl PBS to the top of each agar block and gently pipetting. Liquid was removed by inversion, and the wash was repeated. Aggregate cells were then treated by pipetting 125 μl of 24 mM sodium chlorate (final concentration, 10 mM), 125 μl of 96 μg ml⁻¹ tobramycin (final concentration, 40 μg ml⁻¹), or 62.5 μl of 48 mM chlorate and 62.5 μl of 192 μg ml⁻¹ tobramycin in combination (10 mM and 40μg m⁻¹ final concentrations, respectively) on the top of the agar block. All chlorate and tobramycin solutions were made in LB, and 125 μl LB was pipetted on the top of untreated control agar blocks. Treated and untreated samples were incubated at 37° C. in a humidified chamber, with shaking at 220 rpm for 6 hr. After incubation, samples were inverted to remove liquid, and cells were stained using a BacLight LIVE/DEAD bacterial viability staining kit (Thermo Fisher Scientific number L7012). One hundred twenty-five microliters of staining solution (1 μl 3.34 mM SYTO 9, 1 μl 20 mM propidium iodide, 123 μl dH₂O) was added to each sample, and samples were incubated at room temperature on a VWR variable-speed rocker at the highest speed for 90 min before being imaged.

### Confocal Microscopy, Image Analysis, and Staining Quantification.

Before being imaged, ABBA samples were inverted to remove the staining solution, and 75 μl of a 1:50 dilution of 5-μm fluorescent beads (Spherotech; CFP-5045-2) was added to each sample. Excess liquid was removed by wicking, and beads were used to mark the surface of each sample. ABBA aggregates were imaged using a Leica TCS SPE confocal microscope with an ACS APO 0.3-numeric-aperture/10× objective. The agar surface was determined by visualizing fluorescent beads using a 405-nm solid-state laser for excitation, with data collected from 425 to 475 nm. LIVE/DEAD data were collected using a 488-nm solid-state laser for excitation, with emission collected at 510 to 550 nm and 610 to 650 nm for SYTO 9 and propidium iodide, respectively. Images were collected from three distinct locations near the center of each well as 500-μm z-stacks (50 slices total, 10-μm step size). z-stacks were collected in 8-bit mode with a scan format of 512 by 512 pixels (for quantification) or 1,024 by 1,024 pixels (for representative images) and line averaging of 2. Representative images were chosen from 6 independent experiments.

To quantify ABBA staining, images were loaded in Fiji (92), and background was subtracted from each image using the rolling ball method with a radius of 100 pixels. A threshold was applied to all images to exclude pixels with a value of <20, and total pixel intensity for both stains was determined for each image using the RawIntDen value in the Integrated Density measurement function. The proportion of sensitive cells was determined for each image (at each depth) by dividing the propidium iodide integrated density by the sum of the propidium iodide- and SYTO 9-integrated densities. ABBA staining quantifications are averages from 6 independent experiments (3 technical replicates per independent experiment).

### Oxygen Profiling.

Oxygen profiles were determined from treated ABBA samples (12-hr growth and 6-hr treatment incubations; see above). ABBA samples were kept in a 37° C. water bath during oxygen profiling experiments. Oxygen profiles were measured using a Clark-type amperometric electrode witha 25-μm-tip diameter that was connected to a picoampere amplifier in a multimeter (Unisense, Denmark), as described previously [28]. Briefly, the microsensor was calibrated using a 37° C. oxygen-free solution (0.1 M sodium hydroxide, 0.1 M sodium ascorbate) to obtain a zero-point reading and a 37° C. air-saturated, 1% salt solution corresponding to 199.4 μM oxygen. The microsensor was manipulated via a motorized micromanipulator and was positioned 125 to 150 μm above the air-agar interface prior to data collection. The air-agar interface (depth=0) was defined as the depth at which microsensor values decreased by >0.5% from its baseline value and was determined by moving the microsensor in 10-μm incremental steps. Profiling data were acquired using SensorTrace Pro 3.1.3 software, with which oxygen was measured at intervals of 25 μm for a total depth of 700 μm. Measuring time at each depth was set at 3 s, with 2 s between measuring points. Oxygen profile data are averages from 3 independent experiments (3 technical replicates per independent experiment).

### Example 1

### Chlorate Kills P. aeruginosa Cultures Displaying Physiological Tobramycin Tolerance

Because tobramycin is most effective at killing aerobically growing P. aeruginosa cells [29], whereas chlorate is predicted to target cells containing Nar [26], whether these compounds were effective under different conditions of oxidant exposure was determined. High-density P. aeruginosa cultures (˜10⁹CFU ml⁻¹) were incubated with and without drugs under oxic conditions, anoxic conditions with 40 mM nitrate, or anoxic conditions without nitrate (no terminal electron acceptor) for 4 h, after which viability was determined (FIG. 1).

FIG. 1 shows chlorate kills oxidant-starved P. aeruginosa cells displaying physiological tolerance to tobramycin. Viable-cell plate counts from P. aeruginosa cultures that were incubated for 4 hr without (untreated) or with 40 μg/ml tobramycin, 10 mM chlorate, 40 μg/ml tobramycin plus 10 mM chlorate, or 10 mM chlorite. Cultures were incubated with these compounds under oxic conditions (black), anoxic conditions with 40 mM nitrate (dark gray), or anoxic conditions without nitrate (light gray). Data show the means of results of 9 biological replicates from 3 independent experiments, and error bars indicate standard errors.

As predicted, oxic cultures are sensitive to tobramycin. Anoxic cultures are sensitive to tobramycin when supplied with nitrate but tolerant in the absence of an electron acceptor. Conversely, chlorate does not kill oxic cultures or anoxic cultures supplied with nitrate, while anoxic cultures lacking nitrate are sensitive to chlorate. These findings are consistent with those of prior studies demonstrating that cells require a minimum membrane potential for tobramycin uptake and efficacy [30] [31], which can be established by oxygen or nitrate respiration.

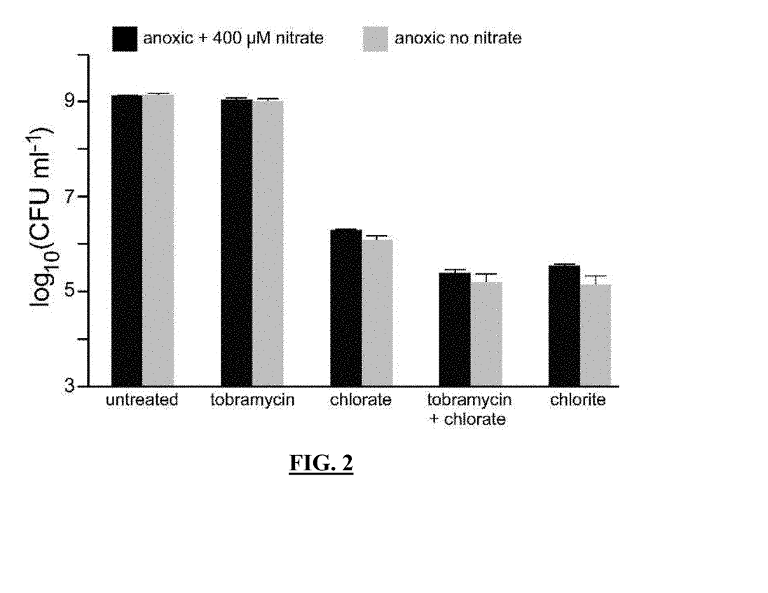

Although anoxic cultures supplied with nitrate utilize Nar, these cells are chlorate tolerant (FIG. 1). However, nitrate concentrations in these experiments were 4-fold higher than chlorate concentrations. When nitrate is provided to anoxic cultures at low concentrations that approximate those found in CF patient sputum or chronic wounds (400 μM nitrate) [32] [25], tobramycin and chlorate lethality is indistinguishable from that in cultures lacking nitrate (see FIG. 2). This can be attributed to the fact that under these conditions, 400 μM nitrate is completely consumed in a short time period (see FIG. 3B).

Combined chlorate and tobramycin treatment under oxic conditions or anoxic conditions plus nitrate results in killing similar to that of tobramycin treatment alone, but combined treatment yields greater death under anoxic conditions (FIG. 1) (0.9 log₁₀ CFU ml⁻¹ more death with the combined treatment than with chlorate treatment; t test, P <0.0001).

Lastly, the sensitivity to chlorite, the predicted product of Nar-mediated chlorate reduction was investigated. Anoxic cultures are much more sensitive to chlorite than oxic cultures (FIG. 1). The mechanism of chlorite toxicity is not fully understood, although it and other reactive chlorine species are known to oxidize amino acids [33], inducing cell death via protein aggregation [34]. Cells with sufficient access to a respiratory oxidant may be better equipped to handle this stress because they have more energy for repair [27] [35].

### Example 2

### Chlorate Toxicity Requires Chlorate Consumption and is Protected by Respiration

To determine whether chlorate reduction is linked to cell death, cell viability and chlorate concentrations in oxic cultures over 72 hr were monitored. Under these conditions, chlorate concentrations are stable, and likewise, there is no chlorate-associated cell death (FIG. 3A). In anoxic cultures supplemented with 40 mM nitrate, nitrate is consumed quickly and undetectable within 8 hr (FIG. 3B). During nitrate consumption, chlorate is consumed at a seemingly constant rate, although much more slowly than nitrate consumption. Chlorate-associated cell death does not occur until nitrate is consumed (dashed line in FIG. 3B). This result suggests that anaerobic respiration may protect cells from chlorate toxicity, perhaps by supplying energy required for chlorite detoxification or repair.

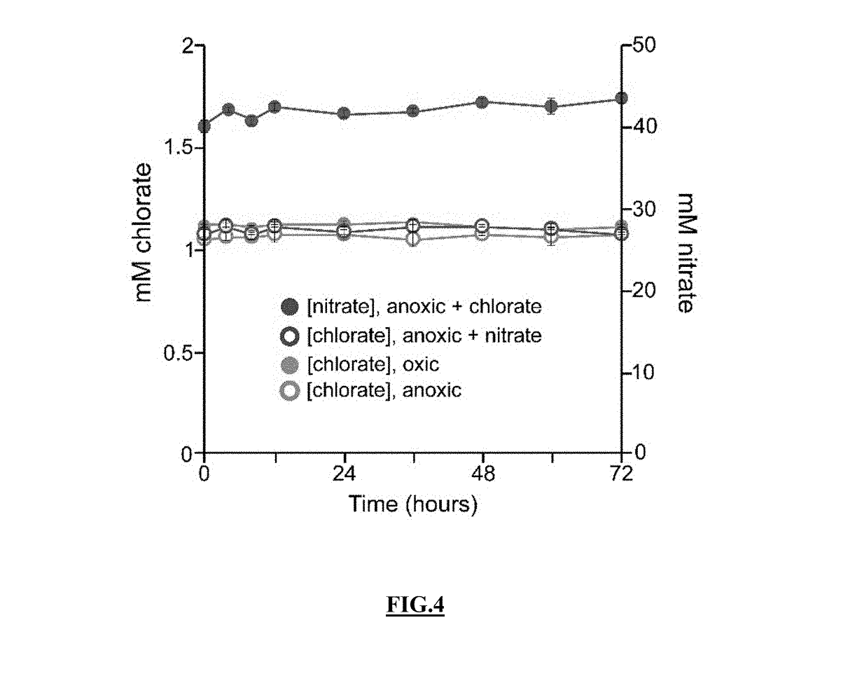

Changes in chlorate and nitrate concentrations can be attributed to cell activity because concentrations of these compounds are stable in abiotic controls (FIG. 4). Further, assuming a 1:1 stoichiometry of chlorate-chlorite converted by Nar, chlorite concentrations exceed our experimental detection limit (1 μM) at the observed rates of chlorate consumption. However, chlorite was not detected in any of the experiments, potentially because chlorite reacts with intracellular components before measurable concentrations can accumulate. Similarly, other reactive chlorine species are known to react quickly with biomolecules [33].

### Example 3

### Nar Genes are Required for Chlorate Consumption and Toxicity

Having shown a correlation between chlorate consumption during oxidant starvation and cell death, experiments were carried out to test whether Nar is required for chlorate consumption and toxicity.

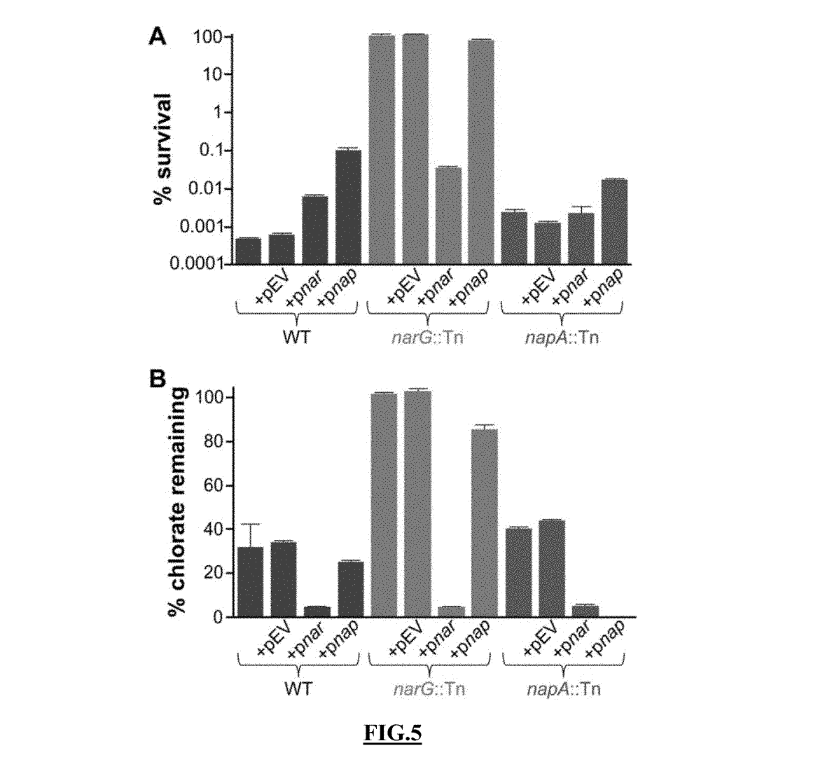

In these experiments, the wild type (WT) and a narG transposon mutant were used and complemented with narGHJI (encoding Nar structural subunits and assembly proteins) carried by an arabinose-inducible vector or with an empty vector. Because narG mutants cannot grow anaerobically, all strains were grown aerobically, washed and resuspended in fresh medium containing or lacking chlorate, and moved to an anaerobic chamber to adapt to anoxia. Viable-cell plate counts were determined after a 72-hr incubation. While oxic WT cultures are not sensitive to chlorate (FIG. 1), anoxia-adapted cultures show a 5-log decrease in viable-cell counts compared to untreated cultures (FIG. 5A). The narG mutant, however, is resistant to chlorate; complementation with the narGHJI genes restores chlorate sensitivity to the narG mutant, while the empty vector has no effect (FIG. 5A). Likewise, chlorate concentrations are stable in narG mutant cultures over the course of the experiment, while chlorate concentrations decrease in WT and narG mutant-complemented cultures (FIG. 5B). This demonstrates that Nar is necessary and sufficient for chlorate reduction and its associated cell death.

Because P. aeruginosa can also reduce nitrate with the periplasmic nitrate reductase Nap [2], experiments were also carried out to test whether Nap also contributes to chlorate sensitivity. napA insertion mutant strains consume 9% less chlorate than WT strains (with and without empty vector strains; t test, P=0.02) and have a 3-fold-higher percentage of survival (with and without empty vector strains; t test, P=0.004). This suggests that periplasmic Nap may play a small role in chlorate reduction, which differs from findings of a prior report stating that this enzyme is incapable of chlorate reduction [36]. When nap genes are overexpressed in a narG mutant background, 15% of the chlorate is consumed. Surprisingly, this is associated with very little cell death (78% survival). Indeed, all strains overexpressing nap genes consume more chlorate yet survive better than their empty vector counterparts (t test, all P <0.02). Similarly, the WT strain overexpressing nar genes consumes more chlorate than its empty-vector counterpart (t test, P <0.0001), yet survives better (t test, P =0.002) (FIG. 5A). The relationship between chlorate reduction and death, thus, appears to be nuanced and may be influenced both by the cellular location of the chlorate reduction machinery and by the chlorate reduction rate.

### Example 4

### The ΔlasR Strain has Increased Rates of Nitrate Respiration and Increased Chlorate Sensitivity

Because P. aeruginosa lasR mutants have increased rates of Nar-mediated nitrate respiration under oxic and hypoxic conditions, it is hypothesized that such mutants might be particularly chlorate sensitive. To test this, the levels of growth and nitrate consumption of WT and ΔlasR strains were compared under different conditions (FIG. 6).

In FIG. 6, cell density (OD₅₀₀, filled circles) and nitrate concentrations (open circles) were monitored in the WT and ΔlasR mutant cultures growing under oxic conditions (A), oxic conditions with 40 mM nitrate (B), and anoxic conditions with 40 mM nitrate (C). (D) The WT and ΔlasR cultures were incubated without (squares) or with (filled circles) 1 mM chlorate under anoxic conditions for 72 h, over which time viable-cell counts (solid lines) and chlorate concentrations (dashed lines) were monitored. Data from all experiments show the means of results from three biological replicates, and error bars indicate standard errors.

The WT and the ΔlasR mutant grow similarly under oxic conditions (FIG. 6A), but upon addition of 40 mM nitrate, the ΔlasR mutant grows more quickly than the WT during late exponential/early stationary phase, though both cultures ultimately reach the same maximum cell density (FIG. 6B). This increased growth rate correlates with increased nitrate consumption by the ΔlasR mutant, which consumes all supplemented nitrate, whereas the WT consumes only 12% over 36 hr (FIG. 6B).

During anaerobic growth with 40 mM nitrate, the ΔlasR mutant also consumes nitrate more quickly than the WT and does so to completion, whereas the WT consumes only 50% over 24 hr (FIG. 6C). Here, rapid and complete nitrate consumption allows anoxic ΔlasR cultures to grow faster and achieve higher cell densities than those of the WT.

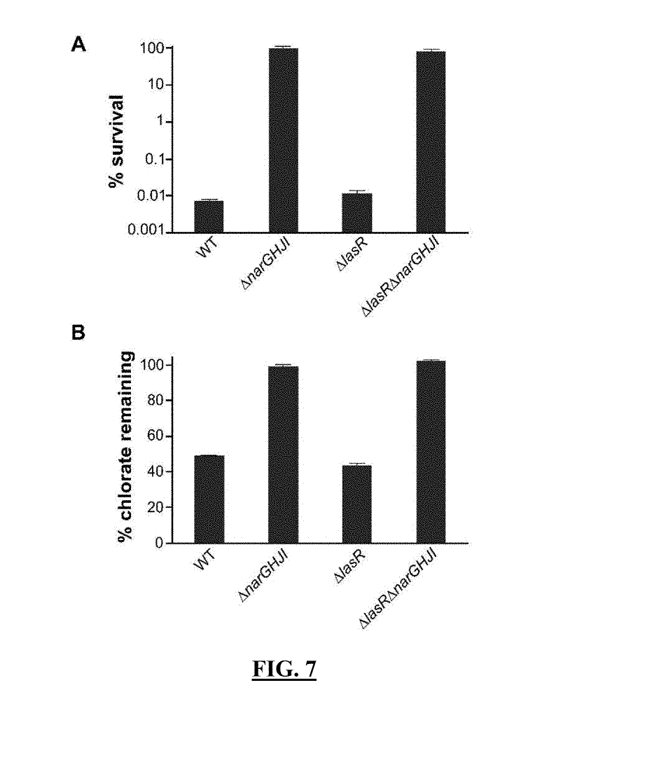

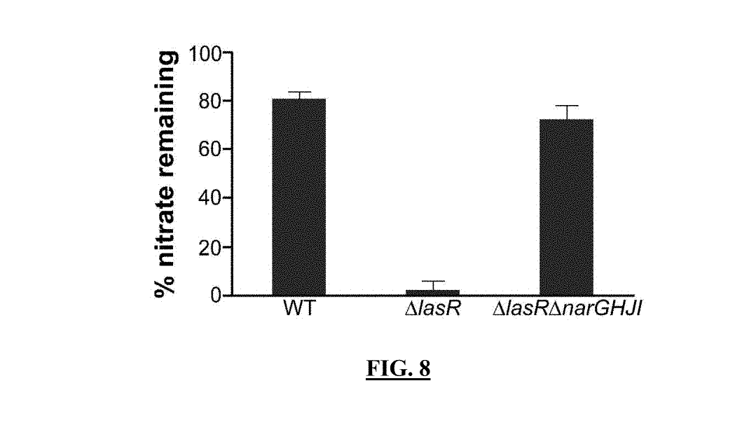

Finally, when incubated under anoxic conditions with chlorate, the ΔlasR mutant consumes chlorate more quickly than the WT, which correlates with increased rates of chlorate associated cell death (FIG. 6D). Although similar amounts of chlorate were ultimately consumed by both strains, viable-cell counts were 100-fold lower in ΔlasR cultures at the end of the experiment, demonstrating that lasR mutants are particularly susceptible to anoxic chlorate treatment. In control experiments, it was found that a ΔlasR ΔnarGHJI strain is chlorate tolerant (FIG. 7) and shows WT levels of nitrate utilization (FIG. 8), demonstrating that chlorate sensitivity in the lasR strain is nar dependent.

### Example 5

### Chlorate and Tobramycin Target Distinct Populations in Aggregate Biofilms

To assess whether the findings in planktonic cultures might apply to a biofilm mode of growth that approximates that found in vivo [37], an agar block biofilm assay (ABBA) was used to study aggregate biofilms.

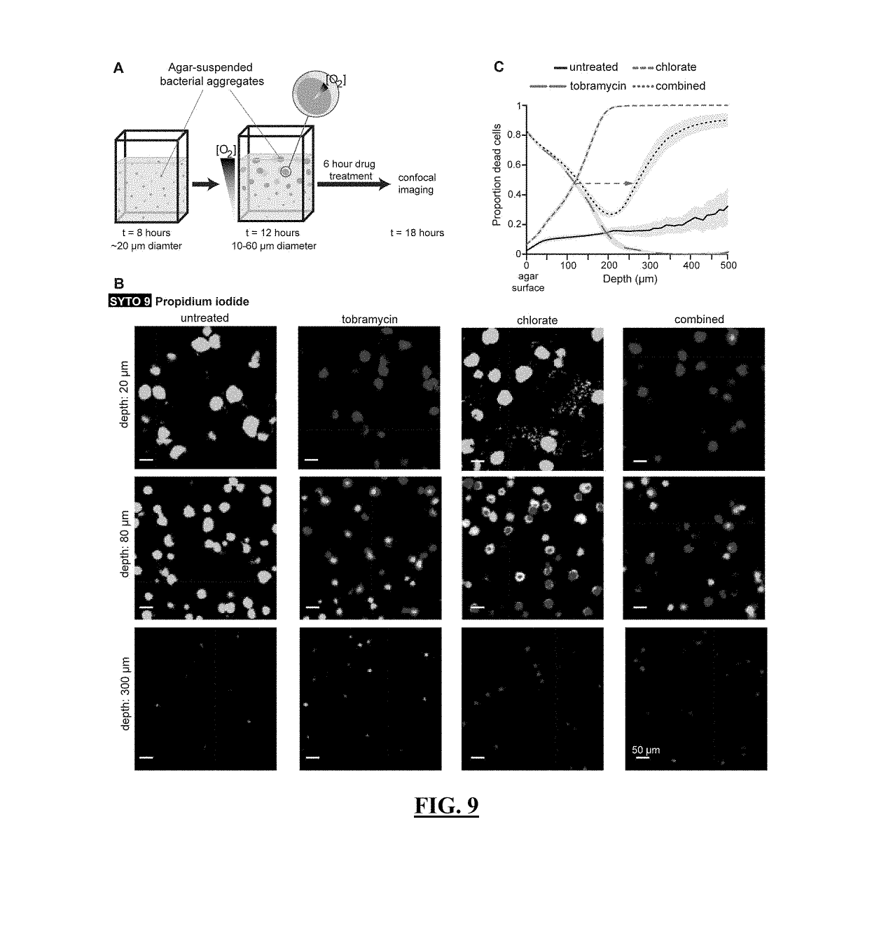

In the ABBA system, an overnight culture is diluted into 0.5% molten agar medium, allowed to solidify, and incubated overnight at 37° C. Agar-suspended cells grow as aggregates, which develop into metabolically distinct populations at different spatial scales. Over time, aggregates near the top of the agar grow more quickly than aggregates deeper in the agar because they consume oxygen that is enriched at the surface, which decreases oxygen availability to deeper aggregates [38] [39] (FIG. 9A). Oxygen gradients can also develop within large aggregates, where cells on the exterior scavenge oxygen before those on the interior can access it [40]. It is predicted that biofilm cells with access to oxygen are tobramycin sensitive and chlorate tolerant but that cells that are oxygen starved are tobramycin tolerant and chlorate sensitive.

P. aeruginosa aggregates were grown overnight in Luria-Bertani medium (LB) agar supplemented with 5 mM nitrate, after which they were treated for 6 hr by pipetting LB with or without tobramycin, chlorate, or both compounds on top of the agar blocks. Following treatment, aggregates were stained with SYTO 9 and propidium iodide (PI) and imaged via confocal microscopy (FIG. 9B). SYTO 9 is membrane permeable and stains all cells, whereas PI is membrane impermeable and, thus, thought to enter nonviable cells with damaged membranes where it displaces SYTO 9 [41]. Though PI can stain viable cells that grow slowly and have a weak membrane potential [42], because a relatively small proportion of cells stain with PI in untreated samples (FIG. 9C), PI- or SYTO 9-stained cells are interpreted as dead or alive in response to drug treatment, respectively.

It was found that cells in surface aggregates are killed by tobramycin, whereas those at depth are tobramycin tolerant (FIG. 9B). The pattern corresponds to the expected profile of oxygen availability [38]. Cells in middepth aggregates display graded sensitivity at the individual aggregate scale, with cells on the exterior being killed and cells on the interior staying alive.

Chlorate targets the opposite population: surface aggregates and the exterior of middepth aggregates are chlorate tolerant, whereas aggregates at depth and the interior of middepth aggregates are killed (FIG. 9B). These findings are consistent with those of our planktonic studies (FIG. 1), where cells with or without access to oxygen were found to be killed by tobramycin or chlorate, respectively.

### Example 6

### Drug Treatment Alters Oxygen Gradients and Drug Sensitivity

Given that tobramycin and chlorate can target and kill distinct, complementary populations within metabolically stratified biofilms, it is anticipated that combined tobramycin and chlorate treatment might compromise all cells within aggregate populations, but paradoxically, this proved not to be the case (FIG. 9B).

In combined-treatment samples, aggregates at the surface were killed by tobramycin and aggregates at depth were killed by chlorate, but middepth aggregates stained similarly to tobramycin-only-treated samples (exterior killed, interior alive). Quantifying these ABBA live/dead profiles, it was found that the combined treatment profile aligns with the tobramycin-only profile for the first 180 μm but that the depths where chlorate kills shifts: the depth at which 50% of the population is killed by chlorate is 162±14 μm (mean±standard error of the mean [SEM]) deeper in combined-treatment samples than in chlorate-only-treated samples (dashed arrow in FIG. 9C). It is speculated that this shift might reflect changes in oxygen availability due to changes in cellular consumption rates, which might explain why combined treatment did not eradicate all cells.

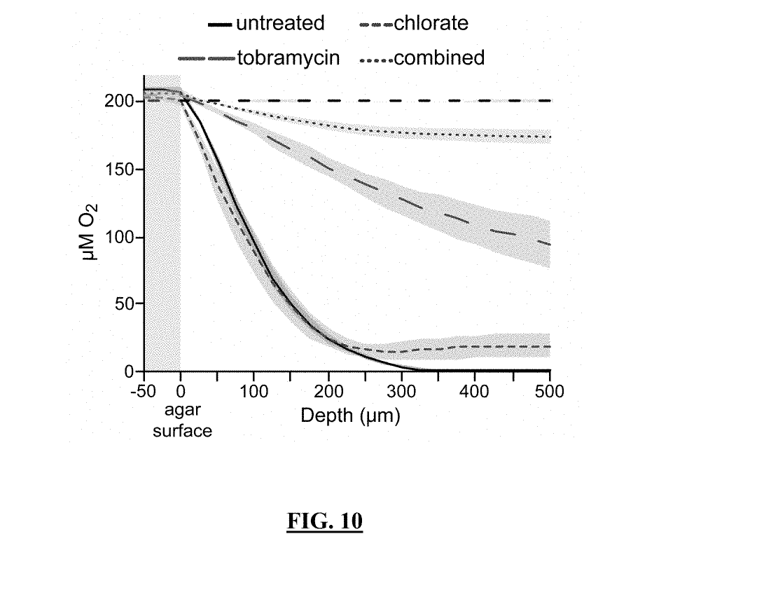

To test this hypothesis, oxygen profiles in ABBA samples were measured (FIG. 10). In untreated samples, oxygen concentrations decrease until anoxia occurs at a depth of about 350 μm. However, tobramycin-treated samples never become anoxic; oxygen concentrations remain at around 50% of atmospheric values at even the greatest depths. This observation supports the hypothesis that the tobramycin-mediated death of surface and exterior cells allows for increased oxygen penetration, leading some populations in combined-treatment samples to shift from a chlorate-sensitive to a -tolerant state (depths of 120 to 220 μm) (FIG. 9C). Increased oxygen penetration also correlates with increased survival in tobramycin-only-treated samples compared to that in untreated samples at depths of >200 μm (FIG. 9C). Oxygen consumption rates for equivalent SYTO-9-stained cells are lower in tobramycin-only-treated (0.27 μM/μm) than in untreated (0.50 μM/μm) samples at depths of 150 to 200 μm (FIG. 10), which may explain why tobramycin-treated cells remain viable at these depths. In chlorate-treated samples, oxygen concentrations decrease until about 200 μm but remain greater than zero (FIG. 10). This is consistent with the finding that most cells at depths of >200 μm are dead in chlorate-treated samples (FIG. 9C). As expected, the combined-treatment samples are relatively oxygen replete (FIG. 10), presumably because few cells survive to respire oxygen.

Though deep aggregates in tobramycin-treated samples are expected to die over prolonged exposure or sequential treatment with chlorate and then tobramycin (or vice versa) is expected to kill all cells given enough time, these experiments are unable to be performed because the ABBA system is not amenable to incubations greater than a day (i.e., a large proportion of cells in the untreated controls stain with PI after ˜20 hr of incubation). Such experiments require a different experimental setup and are priorities for future work.

In summary, described herein are methods, systems, and related compounds and compositions suitable for reducing antibiotic resistance and/or the survivability of Nar (nitrate reductase)-and/or Nap (periplasmic nitrate reductase)-containing bacteria.

The examples set forth above are provided to give those of ordinary skill in the art a complete disclosure and description of how to make and use the embodiments of the compounds, compositions, systems and methods of the disclosure, and are not intended to limit the scope of what the inventors regard as their disclosure. All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by preferred embodiments, exemplary embodiments and optional features, modification and variation of the concepts herein disclosed can be resorted to by those skilled in the art upon the reading of the present disclosure, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all sub-ranges, as well as, all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations which are not specifically disclosed herein.

A number of embodiments of the disclosure have been described. The specific embodiments provided herein are examples of useful embodiments of the disclosure and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In particular, it will be understood that various modifications may be made without departing from the spirit and scope of the present disclosure. Accordingly, other embodiments are within the scope of the following claims.

### REFERENCES

1. Möker, N., C. R. Dean, and J. Tao, Pseudomonas aeruginosa increases formation of multidrug-tolerant persister cells in response to quorum-sensing signaling molecules. J Bacteriol, 2010. 192(7): p. 1946-55.
2. Richardson, D. J., et al., Functional, biochemical and genetic diversity of prokaryotic nitrate reductases. Cell Mol Life Sci, 2001. 58(2): p. 165-78.
3. Altschul S F, M. T., Schaffer A A, Zhang J, Zhang Z, Miller W, Lipman D J., Gapped BLAST and PSI-BLAST: a new generation of protein database search programs. Nucleic Acids Res., 1997. 25(17): p. 14.
4. Smith T F, W. M., Identification of common molecular subsequences. J Mol Biol, 1981. 147(1): p. 3.
5. WR, P., Searching protein sequence libraries: comparison of the sensitivity and selectivity of the Smith-Waterman and FASTA algorithms. Genomics, 1991. 11(3): p. 16.
6. Pearson W R, L. D., Improved tools for biological sequence comparison. Proc Natl Acad Sci USA, 1988. 85(8): p. 5.
7. Johnson L S, E. S., Portugaly E, Hidden Markov model speed heuristic and iterative HMM search procedure. BMC Bioinformatics, 2010. 11(431): p. 8.
8. Myers, E. W. and W. Miller, Optimal alignments in linear space. Computer applications in the biosciences: CABIOS, 1988. 4(1): p. 11-17.
9. Smith, T. F. and M. S. Waterman, Comparison of biosequences. Advances in applied mathematics, 1981. 2(4): p. 482-489.
10. Needleman, S. B. and C. D. Wunsch, A general method applicable to the search for similarities in the amino acid sequence of two proteins. Journal of molecular biology, 1970. 48(3): p. 443-453.
11. Pearson, W. R. and D. J. Lipman, Improved tools for biological sequence comparison. Proceedings of the National Academy of Sciences, 1988. 85(8): p. 2444-2448.
12. Karlin, S. and S. F. Altschul, Methods for assessing the statistical significance of molecular sequence features by using general scoring schemes. Proceedings of the National Academy of Sciences, 1990. 87(6): p. 2264-2268.
13. Karlin, S. and S. F. Altschul, Applications and statistics for multiple high-scoring segments in molecular sequences. Proceedings of the National Academy of Sciences, 1993. 90(12): p. 5873-5877.
14. Moreno-Vivian, C., et al., Prokaryotic nitrate reduction: molecular properties and functional distinction among bacterial nitrate reductases. J Bacteriol, 1999. 181(21): p. 6573-84.
15. D'Argenio, D. A., et al., Growth phenotypes of Pseudomonas aeruginosa lasR mutants adapted to the airways of cystic fibrosis patients. Mol Microbiol, 2007. 64(2): p. 512-33.
16. Hoffman, L. R., et al., Nutrient availability as a mechanism for selection of antibiotic tolerant Pseudomonas aeruginosa within the CF airway. PLoS Pathog, 2010. 6(1): p. e1000712.
17. Smith, E. E., et al., Genetic adaptation by Pseudomonas aeruginosa to the airways of cystic fibrosis patients. Proc Natl Acad Sci USA, 2006. 103(22): p. 8487-92.
18. Hoffman, L. R., et al., Pseudomonas aeruginosa lasR mutants are associated with cystic fibrosis lung disease progression. J Cyst Fibros, 2009. 8(1): p. 66-70.
19. Clark, I. C., et al., Synthetic and Evolutionary Construction of a Chlorate-Reducing Shewanella oneidensis MR-1. MBio, 2015. 6(3): p. e00282-15.
20. Van Alst, N. E., et al., Compensatory periplasmic nitrate reductase activity supports anaerobic growth of Pseudomonas aeruginosa PAO1 in the absence of membrane nitrate reductase. Can J Microbiol, 2009. 55(10): p. 1133-44.
21. Corkery, K., Inhalable drugs for systemic therapy. Respir Care, 2000. 45(7): p. 831-5.
22. Ito, A., et al., Increased antibiotic resistance of Escherichia coli in mature biofilms. Appl Environ Microbiol, 2009. 75(12): p. 4093-100.
23. Bodey, G. P., et al., Infections caused by Pseudomonas aeruginosa. Rev Infect Dis, 1983. 5(2): p. 279-313.
24. Chmiel, J. F. and P. B. Davis, State of the art: why do the lungs ofpatients with cystic fibrosis become infected and why can't they clear the infection? Respir Res, 2003. 4: p. 8.
25. Palmer, K. L., L. M. Aye, and M. Whiteley, Nutritional cues control Pseudomonas aeruginosa multicellular behavior in cystic fibrosis sputum. J Bacteriol, 2007. 189(22): p. 8079-87.
26. Palmer, K. L., S. A. Brown, and M. Whiteley, Membrane-bound nitrate reductase is required for anaerobic growth in cystic fibrosis sputum. J Bacteriol, 2007. 189(12): p. 4449-55.
27. Basta, D. W., M. Bergkessel, and D. K. Newman, Identification of Fitness Determinants during Energy-Limited Growth Arrest in Pseudomonas aeruginosa. MBio, 2017. 8(6).
28. Cowley, E. S., et al., Pediatric Cystic Fibrosis Sputum Can Be Chemically Dynamic, Anoxic, and Extremely Reduced Due to Hydrogen Sulfide Formation. MBio, 2015. 6(4): p. e00767.
29. Williamson, K. S., et al., Heterogeneity in Pseudomonas aeruginosa biofilms includes expression of ribosome hibernation factors in the antibiotic-tolerant subpopulation and hypoxia-induced stress response in the metabolically active population. J Bacteriol, 2012. 194(8): p. 2062-73.
30. Leviton, I. M., et al., Tobramycin uptake in Escherichia coli membrane vesicles. Antimicrob Agents Chemother, 1995. 39(2): p. 467-75.
31. Mates, S. M., et al., Membrane potential in anaerobically growing Staphylococcus aureus and its relationship to gentamicin uptake. Antimicrob Agents Chemother, 1983. 23(4): p. 526-30.
32. Bernatchez, S. F., et al., Nitric oxide levels in wound fluid may reflect the healing trajectory. Wound Repair Regen, 2013. 21(3): p. 410-7.
33. Pattison, D. I. and M. J. Davies, Absolute rate constants for the reaction of hypochlorous acid with protein side chains and peptide bonds. Chem Res Toxicol, 2001. 14(10): p. 1453-64.
34. Winter, J., et al., Bleach activates a redox-regulated chaperone by oxidative protein unfolding. Cell, 2008. 135(4): p. 691-701.
35. Groitl, B., et al., Pseudomonas aeruginosa defense systems against microbicidal oxidants. Mol Microbiol, 2017. 106(3): p. 335-350.
36. Hochstein, L. I. and G. A. Tomlinson, The enzymes associated with denitrification. Annu Rev Microbiol, 1988. 42: p. 231-61.
37. Bjarnsholt, T., et al., The in vivo biofilm. Trends Microbiol, 2013. 21(9): p. 466-74.
38. Costa, K. C., et al., Pyocyanin degradation by a tautomerizing demethylase inhibits Pseudomonas aeruginosa biofilms. Science, 2017. 355(6321): p. 170-173.
39. Sonderholm, M., et al., Tools for studying growth patterns and chemical dynamics of aggregated Pseudomonas aeruginosa exposed to different electron acceptors in an alginate bead model. NPJ Biofilms Microbiomes, 2018. 4: p. 3.
40. Stewart, P. S. and M. J. Franklin, Physiological heterogeneity in biofilms. Nat Rev Microbiol, 2008. 6(3): p. 199-210.
41. Stocks, S. M., Mechanism and use of the commercially available viability stain, BacLight. Cytometry A, 2004. 61(2): p. 189-95.
42. Teal, T. K., et al., Spatiometabolic stratification of Shewanella oneidensis biofilms. Appl Environ Microbiol, 2006. 72(11): p. 7324-30.

1. A method to interfere with viability of Nar-and/or Nap-containing bacteria in a medium, the method comprising
contacting the Nar-containing bacteria with an effective amount of chlorate, alone or in combination with an antibiotic and/or other antimicrobial for a time and under conditions to reduce survivability and/or antibiotic resistance of the bacteria.

2. The method of claim 1, wherein the chlorate is in an amount between 0.001 mM and 200 mM.

3. The method of claim 1, wherein the Nar-and/or Nap-containing bacteria is bacteria containing a NarG protein subunit having at least 80% query coverage and at least 50% sequence similarity with reference to SEQ ID NO: 1.

4, The method of claim 1, wherein the Nar-and/or Nap-containing bacteria is a bacteria containing a NapA protein subunit having at least 85% query coverage and at least 55% sequence similarity compared to SEQ ID NO: 6.

5. The method of claim 1, wherein the antibiotic is selected from the group consisting of Amoxicillin and clavulanic acid, Methicillin, oxacillin, nafcillin, cloxacillin, dicloxacillin, cabenicillin, ticarcillin, piperacillin, mezlocillin, azlocillin, ticarcillin and clavulanic acid, piperacillin and tazobactam, cephalexin, cefdinir, cefprozil, cefaclor, cefuroxime, sulfisoxazole, erythromycin/sulfisoxazole, tobramycin, amikacin, gentamicin, erythromycin, clarithromycin, azithromycin, tetracycline, doxycycline, minocycline, tigecycline, ciprofloxacin, levofloxacin, vancomycin, linezolid, imipenem, meripenem, and aztreonam.

6. The method of claim 1, wherein the Nar-and/or Nap-containing bacteria comprises at least one bacteria selected from the group consisting of Pseudomonas aeruginosa, Staphyloccocus aureus, Proteus spp. Escherichia coli, Propionibacterium acnes, Mycobacterium tuberculosis. Exemplary bacteria in the sense of the disclosure can also include Pseudomonas, Actinomyces israelii, Actinomyces gerencseriae, Brevibacterium, Brevibacterium linens, Coryneform Bacteria, Corynebacterium diphtheria, Nocardia, Bacillus anthracis, Bacillus cereus, Brucella melitensis, Brucella suis, Brucella abortus, Burkholderia cenocepacia, Burkholderia pseudomallei, Pantoea agglomerans, Pectobacterium atrosepticum, Propionibacterium propionicus, Pseudomonas fluorescens, Salmonella enterica, Shigella species, Staphylococcus epidermidis, and Streptomyces anulatus.

7. The method of claim 1, further comprising contacting the Nar-containing bacteria with an effective amount of nitrate.

8. The method of claim 7, wherein the nitrate is in an amount between 0.1 mM and 50 mM.

9. The method of claim 1, wherein the medium comprises biofilm.

10. The method of claim 1, wherein the contacting is performed under hypoxic or anoxic condition.

11. The method of claim 1, further comprising contacting the Nar-containing bacteria with a carbon source.

12. A method for treating and/or preventing a bacterial infection by Nar-containing bacteria in an individual, the method comprising
administering to the individual an effective amount of chlorate alone or in combination with an antibiotic and/or other antimicrobial.

13. The method of claim 12, wherein the administering is performed by aqueous solution, patches, lotion, crease, gel, or aerosol.

14. The method of claim 12, wherein chlorate is in the amount between 0.001 mM and 200 mM.

15. The method of claim 12, wherein the antibiotic is selected from the group consisting of Amoxicillin and clavulanic acid, Methicillin, oxacillin, nafcillin, cloxacillin, dicloxacillin, cabenicillin, ticarcillin, piperacillin, mezlocillin, azlocillin, ticarcillin and clavulanic acid, piperacillin and tazobactam, cephalexin, cefdinir, cefprozil, cefaclor, cefuroxime, sulfisoxazole, erythromycin/sulfisoxazole, tobramycin, amikacin, gentamicin, erythromycin, clarithromycin, azithromycin, tetracycline, doxycycline, minocycline, tigecycline, ciprofloxacin, levofloxacin, vancomycin, linezolid, imipenem, meripenem, and aztreonam.

16. The method of claim 12, wherein the Nar-containing bacteria comprises at least one bacterial strain selected from the group consisting of Pseudomonas aeruginosa, Staphyloccocus aureus, Proteus spp. Escherichia coli, Propionibacterium acnes, Mycobacterium tuberculosis. Exemplary bacteria in the sense of the disclosure can also include Pseudomonas, Actinomyces israelii, Actinomyces gerencseriae, Brevibacterium, Brevibacterium linens, Coryneform Bacteria, Corynebacterium diphtheria, Nocardia, Bacillus anthracis, Bacillus cereus, Brucella melitensis, Brucella suis, Brucella abortus, Burkholderia cenocepacia, Burkholderia pseudomallei, Pantoea agglomerans, Pectobacterium atrosepticum, Propionibacterium propionicus, Pseudomonas fluorescens, Salmonella enterica, Shigella species, Staphylococcus epidermidis, and Streptomyces anulatus.

17. The method of claim 12, further comprising administering to the individual an effective amount of nitrate.

18. The method of claim 17, wherein the nitrate is in an amount between 0.1 mM and 50 mM.

19. An antimicrobial comprising one or more chlorates in an amount suitable to reduce antibiotic resistance and/or survivability of Nar-containing bacteria.

20. A system to interfere with viability of Nar-containing bacteria in a medium, the system comprising
one or more chlorates, one or more antibiotics and/or one or more other antimicrobials,
for concurrent combined or sequential use in the method of claim 1.

21. A system for treating and/or preventing a bacterial infection by a Nar-containing bacteria in an individual, the system comprising
one or more chlorates, one or more antibiotics and/or one or more other antimicrobials,
for concurrent combined or sequential use in the method of claim 12.
